(19) United States
(12) Patent Application Publication
(10) Pub. No.: US 2012/0270884 A1
(43) Pub. Date: Oct. 25, 2012

(54) Methods of Treating Aneurysmal Dilatation, Blood Vessel Wall Weakness and Specifically Abdominal Aortic and Thoracic Aneurysm Using Matrix Metalloprotease-2 Inhibitors

(75) Inventor: Alastair J.J. Wood, New York, NY (US)

(73) Assignee: SYMPHONY EVOLUTION, INC., Rockville, MD (US)

(21) Appl. No.: 13/497,726

(22) PCT Filed: Sep. 30, 2010
(86) PCT No.: PCT/US10/50907

§ 371 Date: Jun. 20, 2012

Related U.S. Application Data
(60) Provisional application No. 61/247,843, filed on Oct. 1, 2009.

(51) Int. Cl. A61K 31/495; A61P 9/00

(52) U.S. Cl. 514/255.01

(57) ABSTRACT

The present invention provides methods of treating aneurysmal dilatation, blood vessel wall weakness, and specifically abdominal aortic aneurysm and thoracic aneurysm by inhibiting MMPs and ADAM-IO. Such compounds are useful in the in vitro study of the role of MMPs and ADAM-10 (and its inhibition) in biological processes. The present invention also comprises pharmaceutical compositions comprising one or more MMPs or ADAM-10 inhibitors according to the invention in combination with a pharmaceutically acceptable carrier. Such compositions are useful for the treatment of aneurysmal dilatation or blood vessel wall weakness, for example abdominal aortic aneurysm and thoracic aneurysm. The invention also comprises methods of treating aneurysmal dilatation or blood vessel wall weakness, for example abdominal aortic aneurysm and thoracic aneurysm utilizing the compounds of the invention in conjunction with inhibitors of angiotensin II, including angiotensin II receptor blockers and angiotensin converting enzyme inhibitors, and cyclophillin inhibitors.

### CROSS-REFERENCE TO RELATED APPLICATIONS

This application claims the benefit of priority to U.S. Provisional Application 61/247,843, filed Oct. 1, 2009, which is hereby incorporated by reference in its entirety.

### BACKGROUND OF THE INVENTION

1. Field of the Invention

The present invention is in the field of methods of use of agents that inhibit matrix metalloproteases (MMPs) and methods of treatment of aneurysmal dilatation or blood vessel wall weakness, including abdominal aortic aneurysm and thoracic aneurysm.

2. Summary of the Related Art

Cell-cell interactions play an important role in regulating cell fate decisions and pattern formation during the development of multicellular organisms. One of the evolutionarily conserved pathways that plays a central role in local cell interactions is mediated by the transmembrane receptors encoded by the Notch (N) gene of Drosophila, the lin-12 and glp-1 genes of C. elegans, and their vertebrate homologs (reviewed in Artavanis-Tsakonas, S., et al. (1995) Notch Signaling. Science 268, 225-232), collectively hereinafter referred to as NOTCH receptors. Several lines of evidence suggest that the proteolytic processing of NOTCH receptors is important for their function. For example, in addition to the full-length proteins, antibodies against the intracellular domains of NOTCH receptors have detected C-terminal fragments of 100-120 kd; see, e.g., Fehon, R. G., et al. (1990). Cell 61, 523-534; Crittenden, S. L., et al. (1994). Development 120, 2901-2911; Aster, J., et al. (1994) Cold Spring Harbor Symp. Quant. Biol. 59, 125-136; Zagouras, P., et al. (1995). Proc. Natl. Acad. Sci. U.S.A. 92, 6414-6418; and Kopan, R., et al. (1996). Proc. Natl. Acad. Sci. U.S.A. 93, 1683-1688. However, the mechanism(s) of NOTCH activation have been hitherto largely unknown.

During neurogenesis, a single neural precursor is singled out from a group of equivalent cells through a lateral inhibition process in which the emerging neural precursor cell prevents its neighbors from taking on the same fate (reviewed in Simpson, P. (1990). Development 109, 509-519). Genetic studies in Drosophila have implicated a group of “neurogenic genes” including N in lateral inhibition. Loss-of-function mutations in any of the neurogenic genes result in hypertrophy of neural cells at the expense of epidermis “neurogenic genes” including N in lateral inhibition. Loss-of-function mutations in any of the neurogenic genes result in hypertrophy of neural cells at the expense of epidermis (reviewed in Campos-Ortega, J. A. (1993) In: The Development of Drosophila melanogaster M. Bate and A. Martinez-Arias, eds. pp. 1091-1129. Cold Spring Harbor Press).

Rooke, J., Pan, D. J., Xu, T. and Rubin, G. M. (1996). Science 273, 1227-1231, discloses neurogenic gene family, kuzbanian (kuz). Members of the KUZ family of proteins are shown to belong to the recently defined ADAM family of transmembrane proteins, members of which contain both a disintegrin and metalloprotease domain (reviewed in Wolfsberg, T. G., et al. (1995). J. Cell Biol. 131, 275-278, see also Blobel, C. P., et al. (1992). Nature 356, 248-252, 1992; Yagami-Hiromasa, T., et al. (1995). Nature 377, 652-656; Black, R. A., et al. (1997). Nature 385, 729-733, 1997; and Moss, M. L., et al. (1997). Nature 385, 733-736; see also U.S. Pat. No. 5,922,546 and U.S. Pat. No. 5,935,792).

Genes of the ADAM family encode transmembrane proteins containing both metalloprotease and disintegrin domains (reviewed in Black and White, 1998 Curr. Opin. Cell Biol. 10, 654-659; Wolfsberg and White, 1996 Dev. Biol. 180, 389-401), and are involved in diverse biological processes in mammals such as fertilization (Cho et al., 1998 Science 281, 1857-1859), myoblast fusion (Yagami-Hiromasa et al., 1995 Nature 377, 652-656) and ectodomain shedding (Moss et al., 1997 Nature 385, 733-736; Black et al., 1997 Nature 385, 729-733; Peschon et al., 1998 Science 282, 1281-1284). The Drosophila kuzbanian (kuz) gene represents the first ADAM family member identified in invertebrates (Rooke et al., 1996 Science 273, 1227-1231). Previous genetic studies showed that kuz is required for lateral inhibition and axonal outgrowth during Drosophila neural development (Rooke et al., 1996; Fambrough et al., 1996 PNAS.USA 93, 13233-13238; Pan and Rubin, 1997 Cell 90, 271-280; Sotillos et al., 1997 Development 124, 4769-4779). Specifically, during the lateral inhibition process, kuz acts upstream of Notch (Pan and Rubin, 1997; Sotillos et al., 1997), which encodes the transmembrane receptor for the lateral inhibition signal encoded by the Delta gene. More recently, a homolog of kuz was identified in C. elegans (SUP-17) that modulates the activity of a C. elegans homolog of Notch in a similar manner (Wen et al., 1997 Development 124, 4759-4767).

Vertebrate homologs of kuz have been isolated in Xenopus, bovine, mouse, rat and human. The bovine homolog of KUZ (also called MADM or ADAM 10) was initially isolated serendipitously based on its in vitro proteolytic activity on myelin basic protein, a cytoplasmic protein that is unlikely the physiological substrate for the bovine KUZ protease (Howard et al., 1996 Biochem. J. 317, 45-50). Expression of a dominant negative form of the murine kuz homolog (mkuz) in Xenopus leads to the generation of extra neurons, suggesting an evolutionarily conserved role for mkuz in regulating Notch signaling in vertebrate neurogenesis (Pan and Rubin, 1997). U.S. patent application Ser. No. 09/697,854, to Pan et al., filed Oct. 27, 2000, discloses that mkuz mutant mice die around embryonic day (E) 9.5, with severe defects in the nervous system, the paraxial mesoderm and the yolk sac vasculature. In the nervous system, mkuz mutant embryos show ectopic neuronal differentiation. In the paraxial mesoderm, mkuz mutant embryos show delayed and uncoordinated segmentation of the somites. These phenotypes are similar to those of mice lacking Notch-1 or components of the Notch pathway such as RBP-Jk (Conlon et al, 1995, Development 121, 1533-1545; Oka et al., 1995), indicating a conserved role for mkuz in modulating Notch signaling in mouse development. Furthermore, no visible defect was detected in Notch processing in the kuz knockout animals. In addition to the neurogenesis and somitogenesis defect, mkuz mutant mice also show severe defects in the yolk sac vasculature, with an enlarged and disordered capillary plexus and the absence of large vitelline vessels. Since such phenotype has not been observed in mice lacking Notch-1 or RBP-Jk (Swiatek et al., 1994 Genes Dev 15, 707-719; Conlon et al, 1995; Oka et al., 1995 Development 121, 3291-3301), Pan et al. determined that this phenotype reveals a novel function of mkuz that is distinct from its role in modulating Notch signaling, specifically, that kuz plays an essential role for an ADAM family disintegrin metalloprotease in mammalian angiogenesis.

In view of the important role of KUZ (ADAM-10) in biological processes and disease states, inhibitors of this protein are desirable, particularly small molecule inhibitors.

Matrix metalloproteinases, or MMPs, are endopepitidases that are collectively capable of degrading all kinds of extracellular matrix proteins, but can also process a number of bioactive molecules. MMPs are thought to play a major role in cell proliferation, migration, differentiation, angiogenesis, apoptosis, and host defense. MMPs break down elastin and interstitial collagens, which are important in maintaining the strength and elasticity of the aortic wall.

An aneurysm is a localized, blood-filled dilitation (balloon-like bulge) of a blood vessel caused by disease or weakening of the vessel wall. As the size of an aneurysm increases, there is an increased risk of rupture, which can result in severe hemorrhage or other complications including sudden death. Abdominal aortic aneurysms, which are weaknesses in the abdominal aortic walls, occur in up to 9% of adults older than 65 years of age, and the rupture of these aneurysms accounts for about 15,000 deaths per year in the United States (Weintraub, 2009 NEJM, 361; 11, 1114-1116). Currently, it is the standard practice to aggressively treat hypertension and hyperlipidemia in patients with abdominal aortic aneurysms because these conditions are risk factors for such aneurysms; but such aggressive therapies have little effect on aneurysm growth or rupture.

Studies have suggested that selective inhibition of matrix metalloproteases is important. A number of small molecule matrix metalloprotease inhibitors (MMPI's) have progressed into the clinic for cancer and rheumatoid arthritis, for example. Inhibition of MMP-1 has been implicated as the cause of side effects such as joint pain and tendonitis when unselective TACE inhibitors were employed (see Barlaam, B. et. al. J. Med. Chem. 1999, 42, 4890). As well, clinical trials of broad spectrum MMP inhibitors, such as “Marimastat,” have been hampered due to musculoskeletal syndrome (MSS) which manifests as musculoskeletal pain after a few weeks treatment. Inhibition of MMP-1 has been suggested as having a role in the appearance of MSS. Recent efforts in the field have been directed toward design of “MMP-1 sparing” inhibitors; for example, BA-129566 emerged as a selective inhibitor which reportedly showed no signs of MSS in phase 2 clinical trials (see Natchus, M. G. et. Al. J. Med. Chem. 2000, 43, 4948).

Thus, there is a need for selective matrix metalloprotease inhibitors.

All patents, applications, and publications recited herein are hereby incorporated by reference in their entirety.

### SUMMARY OF THE INVENTION

The invention comprises methods of treating diseases by inhibiting MMPs. Such diseases include aneurysmal dilatation or blood vessel wall weakness, including abdominal aortic aneurysm and thoracic aneurysm, by administering these inhibiting compounds, alone or in combination (simultaneously or serially) with an ACE inhibitor (angiotensin converting enzyme inhibitor), an ARB (angiotensin II receptor blocker), and/or a cyclophilin inhibitor (e.g., cyclosporine A).

The foregoing merely summarizes certain aspects of the invention and is not intended to be limiting.

### BRIEF DESCRIPTION OF THE DRAWINGS

FIG. 1 shows the effect of treatment in mice with increasing doses of the experimental agent via daily gavage on aortic dilation 14 days following isolated aortic elastase perfusion. Results are reported as Mean±SE. Data was compared with ANOVA using Tukey's correction for multiple comparisons among the treatment groups. Significant differences are indicated by a line connecting the two groups with a significance value over the line. All significant (P<0.05) comparisons are shown.

FIG. 2 shows data described in FIG. 1 shown in Box and Whisker plot format. Increasing doses of the experimental affect the median % ΔAD at 14 days following isolated aortic perfusion.

### DETAILED DESCRIPTION OF THE INVENTION

The present invention comprises methods of treatment of aneurysmal dilatation or blood vessel wall weakness, including abdominal aortic aneurysm and thoracic aneurysm, utilizing these inhibitors.

In embodiment 1, the invention comprises a method of treating aneurysmal dilatation and blood vessel wall weakness, including abdominal aortic aneurysms and thoracic aneurysms, comprising administering to a subject a therapeutically effective amount of a compound of structural formula I:

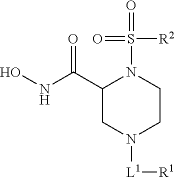

and pharmaceutically acceptable salts, esters, amides, and prodrugs thereof wherein

L¹ is —C(O)—, —S(O)₂—, or —(CH₂)ₙ—;

R¹ is —H, —OR¹¹, —(CH₂)ₙR¹¹, —C(O)R¹¹, or —NR¹²R¹³;
R¹¹, R¹², and R¹³ independently are
   a) R⁵⁰;
   b) saturated or mono- or poly-unsaturated C₅-C₁₄-mono- or fused poly-cyclic hydrocarbyl, optionally containing one or two annular heteroatoms per ring and optionally substituted with one or two R⁵⁰ substituents;
   c) C₁-C₆-alkyl, C₂-C₆-alkenyl, C₂-C₆-alkynyl, or —C(O)H, each of which is optionally substituted with one, two or three substituents independently selected from R⁵⁰ and saturated or mono- or poly-unsaturated C₅-C₁₄-mono- or fused poly-cyclic hydrocarbyl, optionally containing one or two annular heteroatoms per ring and optionally substituted with one, two or three R⁵⁰ substituents;
   or R¹² and R¹³ together with the N to which they are covalently bound, a C₅-C₆ heterocycle optionally containing a second annular heteroatom and optionally substituted with one or two R⁵⁰ substituents;

R² is —R²¹-L²-R²²;
R²¹ is saturated or mono- or poly-unsaturated C₅-C₁₄-mono- or fused poly-cyclic hydrocarbyl, optionally containing one or two annular heteroatoms per ring and optionally substituted with one, two, or three R⁵⁰ substituents;
L² is —O—, —C(O)—, —CH₂—, —NH—, —S(O₂)— or a direct bond;
R²² is saturated or mono- or poly-unsaturated C₅-C₁₄-mono- or fused poly-cyclic hydrocarbyl, optionally containing one or two annular heteroatoms per ring and optionally substituted with one, two, or three R⁵⁰ substituents; and

R⁵⁰ is R⁵¹-L³-(CH₂)ₙ—;
L³ is —O—, —NH—, —S(O)₀₋₂—, —C(O)—, —C(O)O—, —C(O)NH—, —OC(O)—, —NHC(O)—, —C₆H₄—, or a direct bond;
R⁵¹ is —H, C₁-C₆-alkyl, C₂-C₆-alkenyl, C₂-C₆-alkynyl, halo, —CF₃, —OCF₃, —OH, —NH₂, mono-C₁-C₆alkyl amino, di-C₁-C₆alkyl amino, —SH, —CO₂H, —CN, —NO₂, —SO₃H, or a saturated or mono- or poly-unsaturated C₅-C₁₄-mono- or fused poly-cyclic hydrocarbyl, optionally containing one or two annular heteroatoms per ring and optionally substituted with one, two, or three substituents;

wherein n is 0, 1, 2, or 3;

provided that an O or S is not singly bonded to another O or S in a chain of atoms.

In embodiment 2, the invention comprises the method according to embodiment 1 wherein L¹ is —C(O)— or —S(O)₂—.

In embodiment 3, the invention comprises the method according to embodiment 2 wherein L¹ is —C(O)— and R¹ is —OR¹¹ or —(CH₂)ₙR¹¹, —OC₁-C₆alkyl-mono-C₁-C₆alkyl amino, —OC₁-C₆alkyl-di-C₁-C₆alkyl amino, —OC₁-C₆alkyl-N-heterocyclyl, —C₁-C₆alkyl-mono-C₁-C₆alkyl amino, —C₁-C₆alkyl-di-C₁-C₆alkyl amino, or —C₁-C₆alkyl-N-heterocyclyl. In a more specific example, R¹ is C₁-C₆-alkoxy-C₁-C₆-alkoxy; and in a still more specific example R¹ is methoxyethoxy.

In embodiment 4, the invention comprises the method according to embodiment 3 wherein, L¹ is —S(O)₂—, and R¹ is —NR¹²R¹³, —(CH₂)ₙR¹¹, —C₁-C₆alkyl-mono-C₁-C₆alkyl amino, —C₁-C₆alkyl-di-C₁-C₆alkyl amino, or —C₁-C₆alkyl-N-heterocyclyl.

In embodiment 5, the invention comprises the method according to embodiments 3 or 4, wherein L² is —O—.

In embodiment 6, the invention comprises the method according to embodiment 5, R² is phenoxyphenyl wherein each phenyl is optionally substituted with one or two R⁵⁰ substituents. In a more specific example, the R⁵⁰ substituents are halo.

In embodiment 7, the invention comprises the method according to embodiment 6, wherein the saturated or mono- or poly-unsaturated C₅-C₁₄-mono- or fused poly-cyclic hydrocarbyl containing one or two annular heteroatoms per ring is selected from the group consisting of morpholinyl, piperazinyl, homopiperazinyl, pyrrolidinyl, piperidinyl, homopiperidinyl, furyl, thienyl, pyranyl, isobenzofuranyl, chromenyl, pyrrolyl, imidazolyl, isoxazolyl, pyridyl, pyrazinyl, pyrimidinyl, oxadiazolyl, indolyl, quinolinyl, carbazolyl, acrydinyl, and furazanyl, optionally substituted with one or two R⁵⁰ substituents.

In embodiment 8, the invention comprises the method according to embodiment 6, wherein R¹² and R¹³, together with the N to which they are covalently bound, form a heterocycle selected from the group consisting of morpholinyl, piperazinyl, homopiperazinyl, pyrrolidinyl, piperidinyl, homopiperidinyl, pyrrolyl, imidazolyl, isoxazolyl, pyridyl, pyrazinyl, pyrimidinyl, oxadiazolyl, indolyl, quinolinyl, carbazolyl, acrydinyl, and furazanyl, optionally substituted with one or two R⁵⁰ substituents.

In embodiment 9, the invention comprises the method utilizing the compound according to embodiment 1, having the absolute stereochemistry of structural formula II:

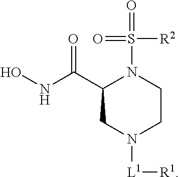

In embodiment 10, the invention comprises the method according to embodiment 1, wherein the compound has the absolute stereochemistry of structural formula III:

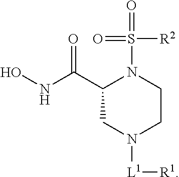

In embodiment 11, the invention comprises the method according to embodiment 1, wherein -L¹-R¹ is selected from Table 1;

TABLE 1

| | | |
|---|---|---|
| —R¹⁴ | 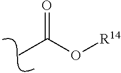 | 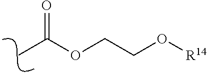 |
| 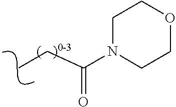 | 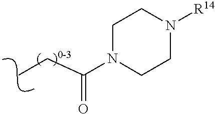 | 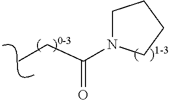 |
| 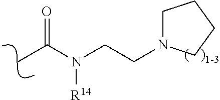 | 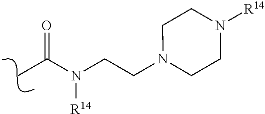 | 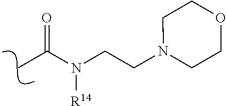 |
| 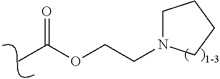 | 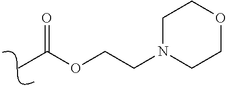 | 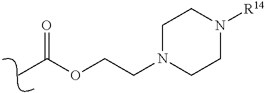 |
| 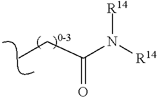 | 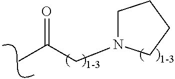 | 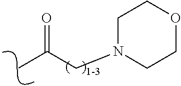 |
| 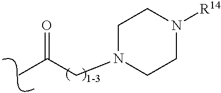 | 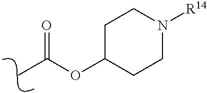 | 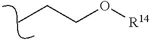 |
| 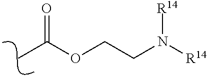 | 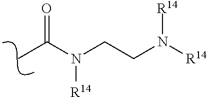 | 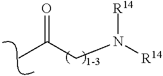 |
| 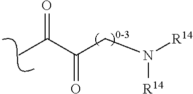 | 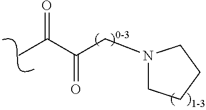 | 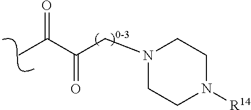 |
| 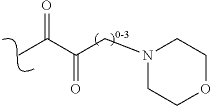 | 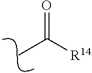 | 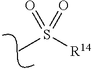 |
| 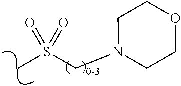 | 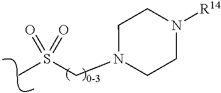 | 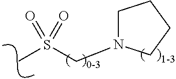 |
| 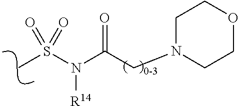 | 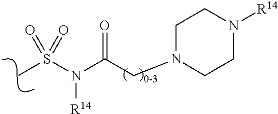 | 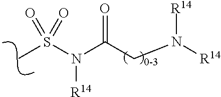 |
| 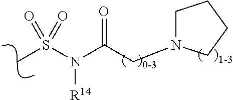 | 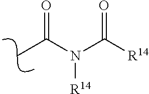 | 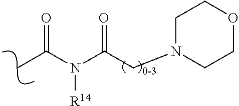 |
| 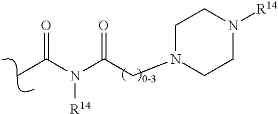 | 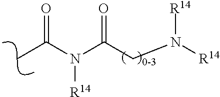 | 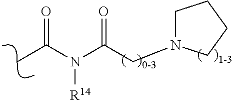 |

wherein each R¹⁴ is independently selected from —H, —(CH₂)₁₋₃CO₂H, alkyl, alkoxy, alkenyl, aryl, heteroaryl, arylalkyl, and heteroarylalkyl;
and R² is selected from Table 2;

TABLE 2

| | | |
|---|---|---|
| 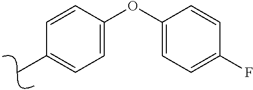 | 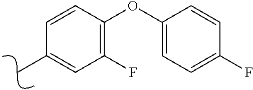 | 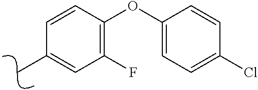 |
| 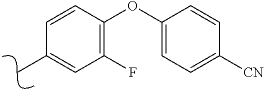 | 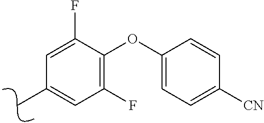 | 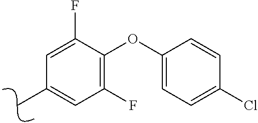 |
| 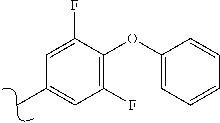 | 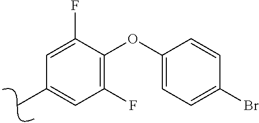 | 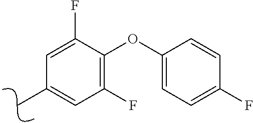 |
| 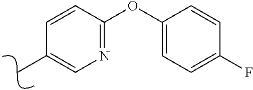 | 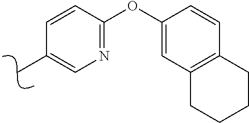 | 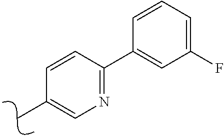 |
| 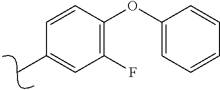 | 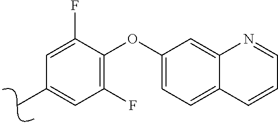 | 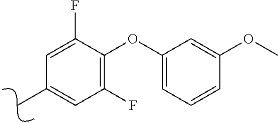 |
| 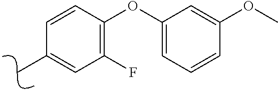 | 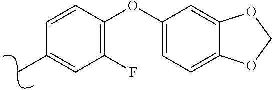 | 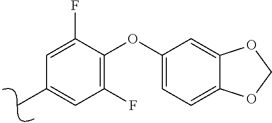 |
| 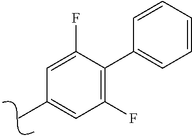 | 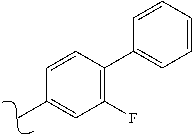 | 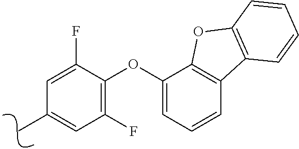 |
| 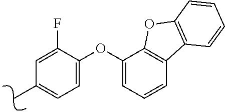 | 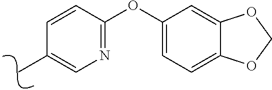 | 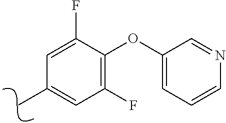 |
| 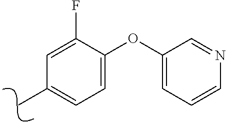 | 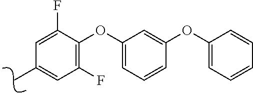 | 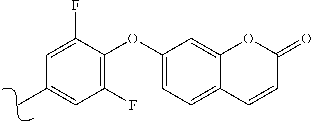 |

In embodiment 12, the invention comprises the method according to embodiment 1, wherein the compound is selected from Table 3:

TABLE 3

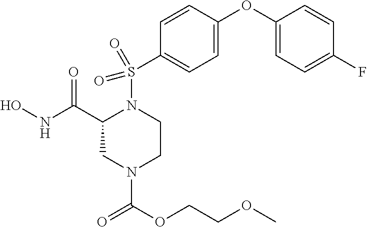

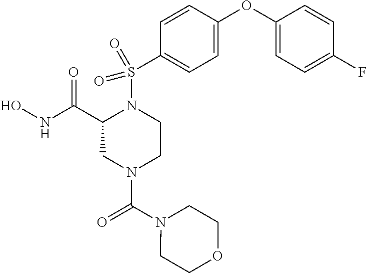

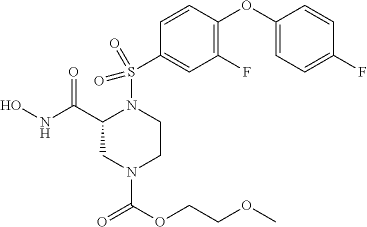

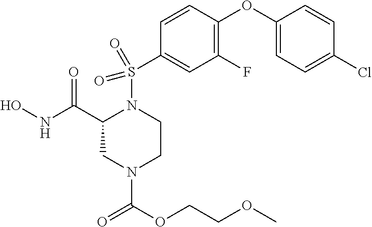

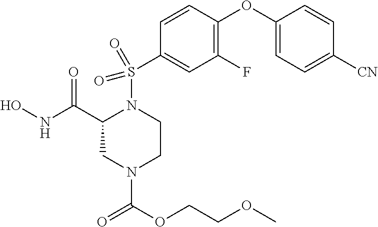

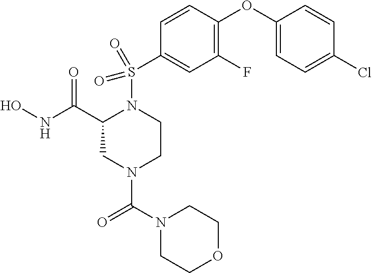

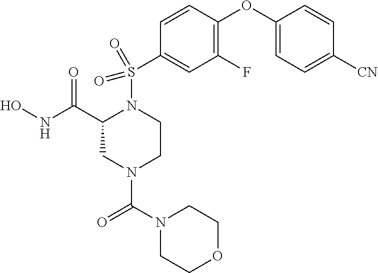

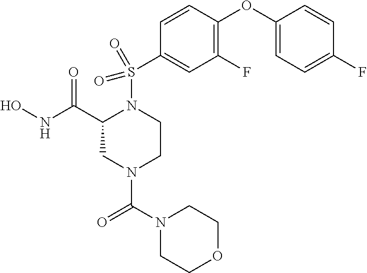

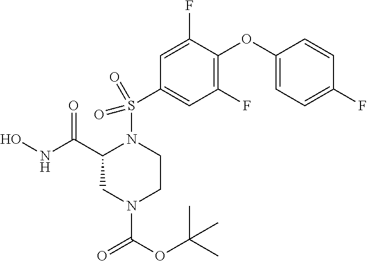

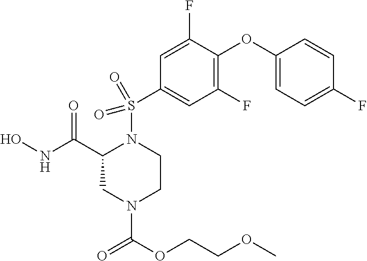

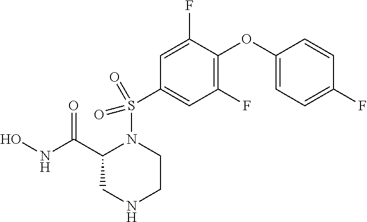

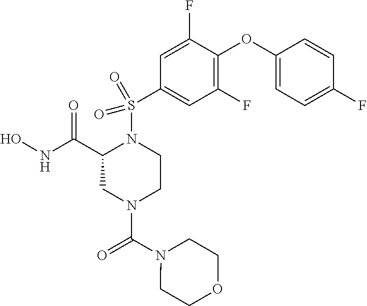

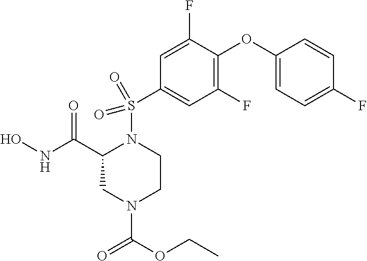

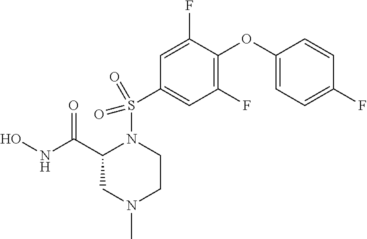

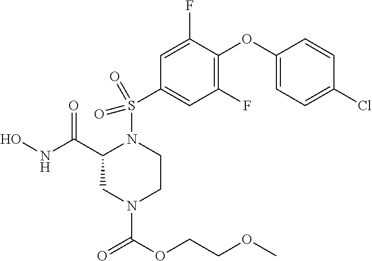

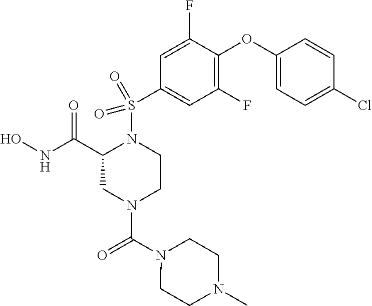

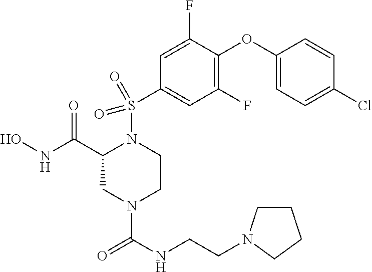

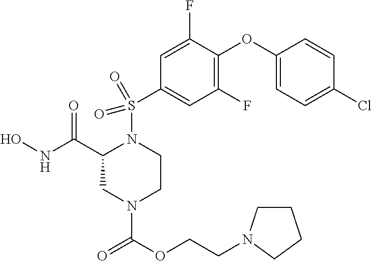

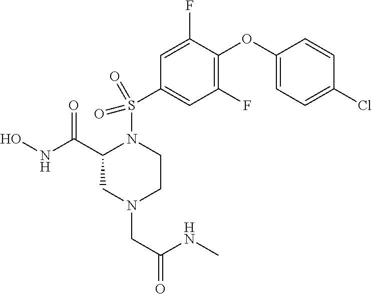

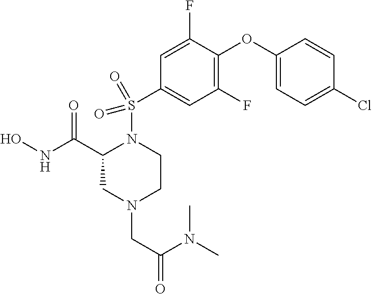

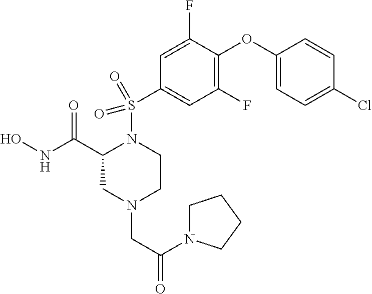

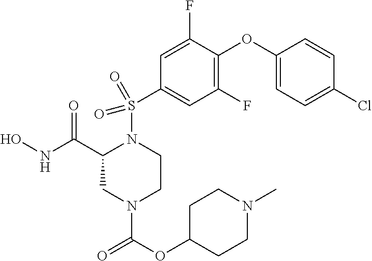

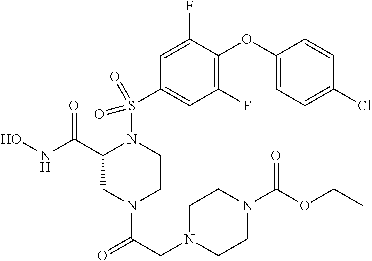

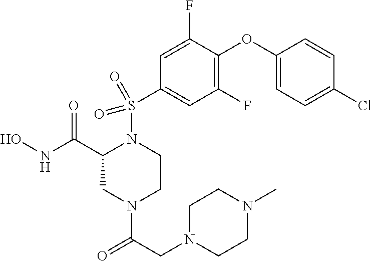

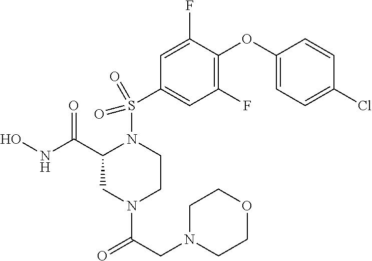

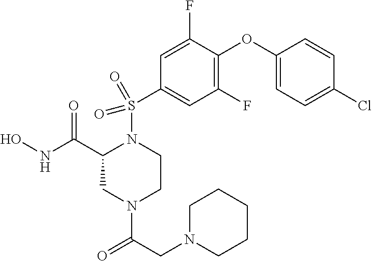

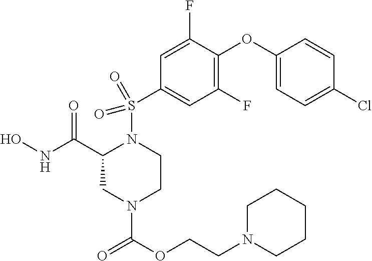

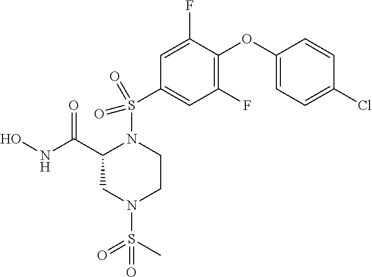

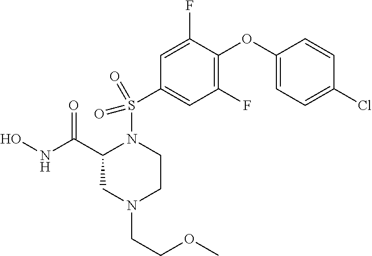

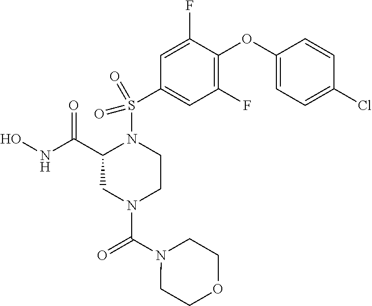

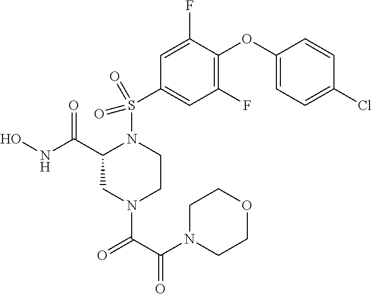

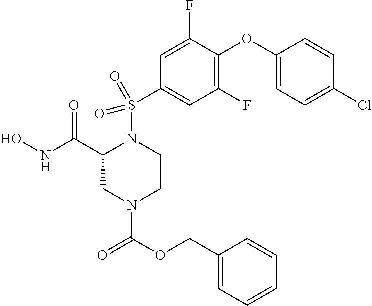

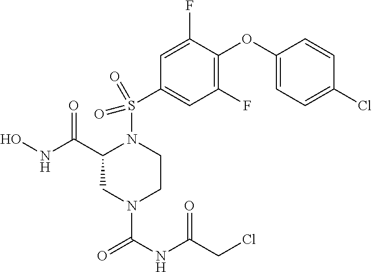

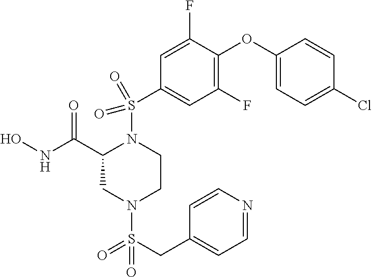

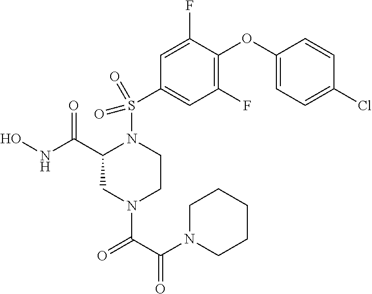

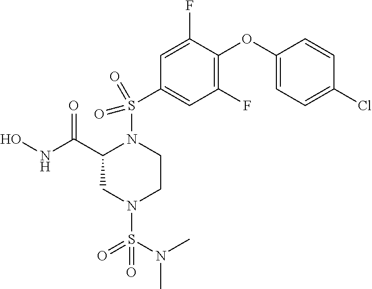

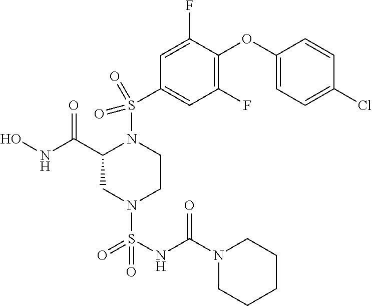

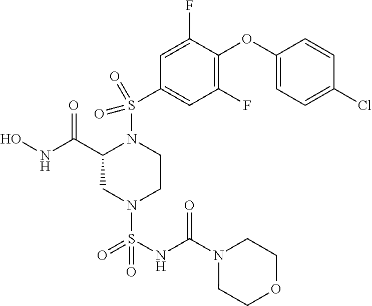

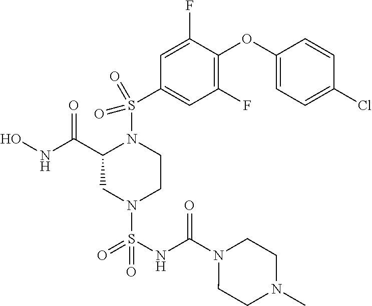

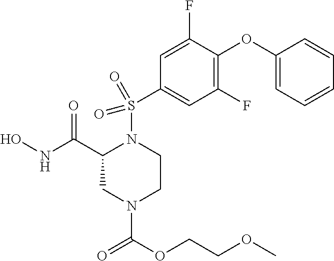

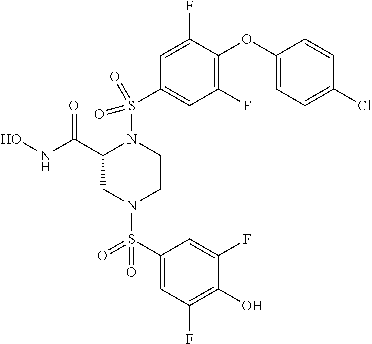

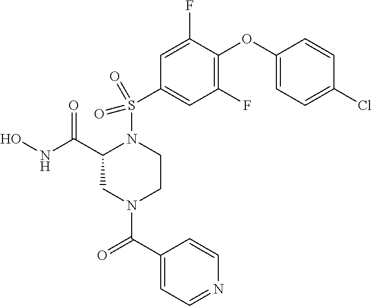

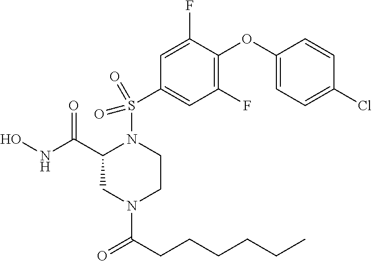

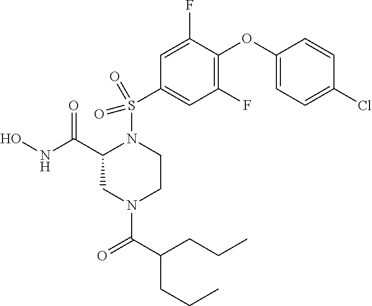

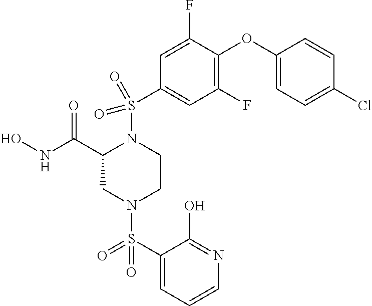

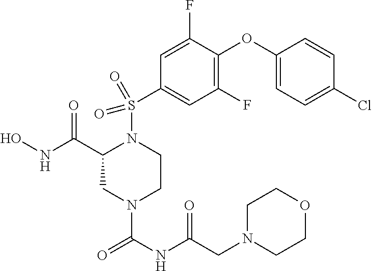

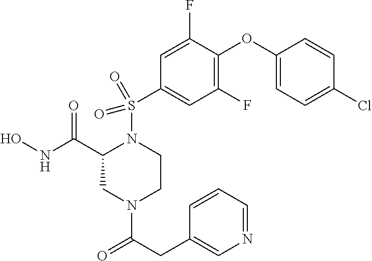

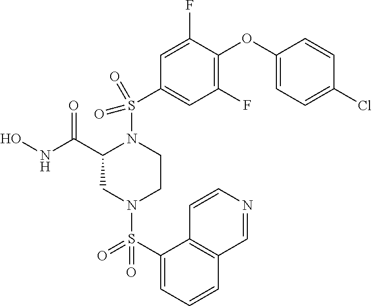

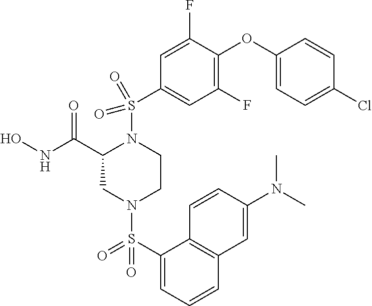

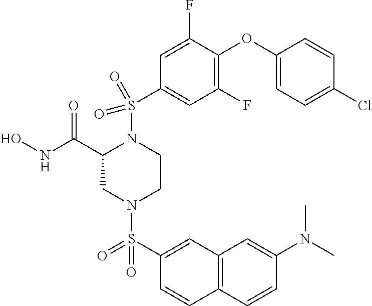

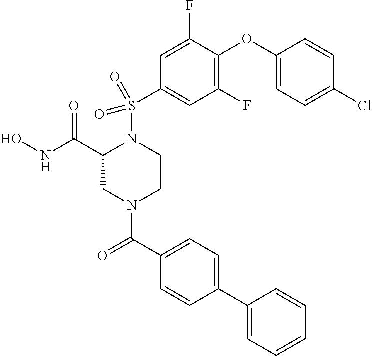

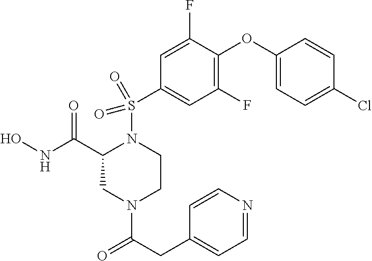

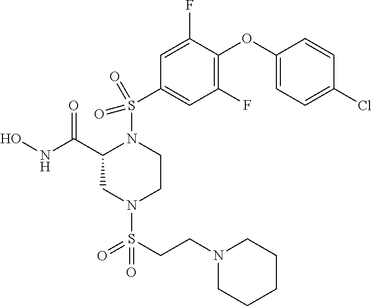

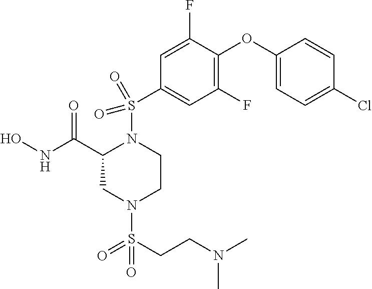

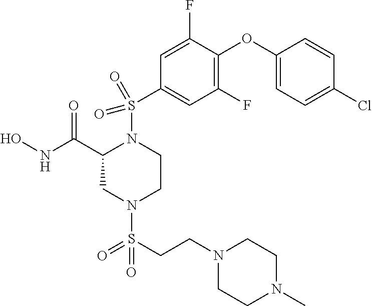

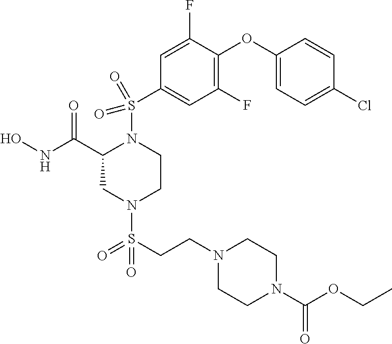

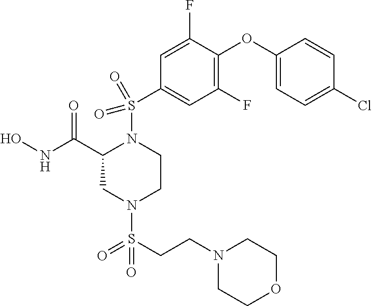

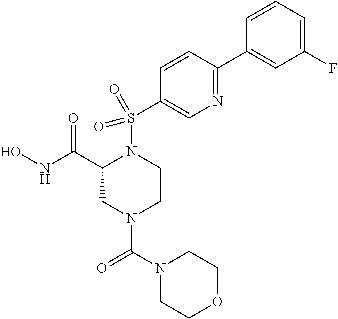

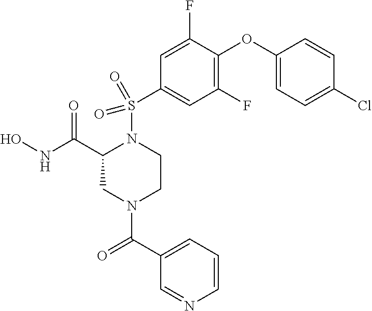

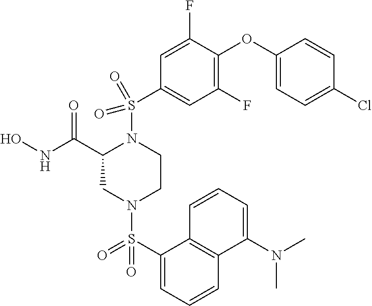

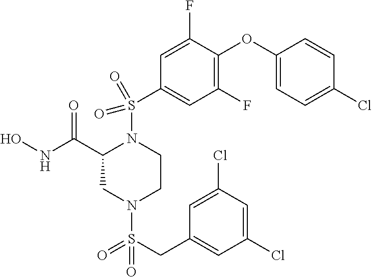

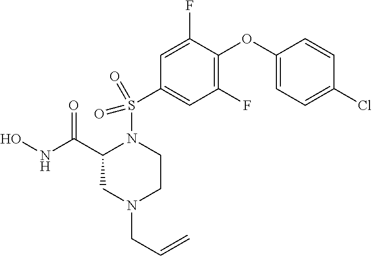

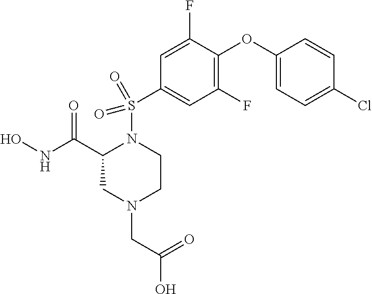

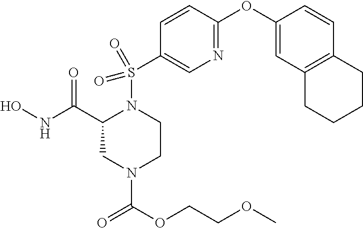

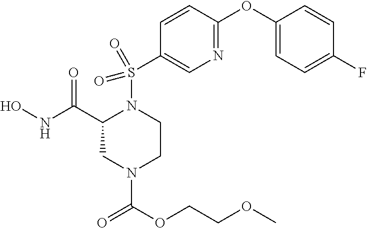

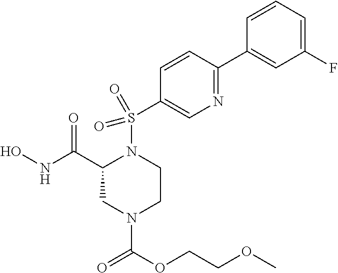

In embodiment 13, the invention comprises a method of treating aneurysmal dilatation or blood vessel wall weakness, including abdominal aortic aneurysm and thoracic aneurysm, comprising administering to a subject with an aneurysmal dilatation or blood vessel wall weakness a therapeutically effective amount of a compound according to formula IV,

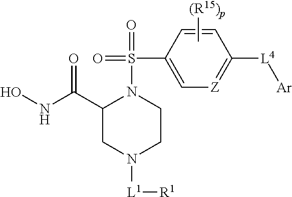

or a pharmaceutically acceptable salt, ester, amide, or prodrug thereof wherein,

Z is —C(R¹⁵)═, —C(H)═, or —N═;

Ar is aryl or heteroaryl, each optionally substituted;

R¹⁵ is fluoro;

p is 0, 1, 2, or 3;

L¹ is —C(O)—, —S(O)₂—, or —(CH₂)ₙ—;

L⁴ is nothing or —O—;

R¹ is —H, —OR¹¹, —(CH₂)ₙR¹¹, —C(O)R¹¹, or —NR¹²R¹³;
R¹¹, R¹², and R¹³ independently are
   d) R⁵⁰;
   e) saturated or mono- or poly-unsaturated C₅-C₁₄-mono- or fused poly-cyclic hydrocarbyl, optionally containing one or two annular heteroatoms per ring and optionally substituted with one or two R⁵⁰ substituents;
   f) C₁-C₆-alkyl, C₂-C₆-alkenyl, C₂-C₆-alkynyl, or —C(O)H, each of which is optionally substituted with one, two or three substituents independently selected from R⁵⁰ and saturated or mono- or poly-unsaturated C₅-C₁₄-mono- or fused poly-cyclic hydrocarbyl, optionally containing one or two annular heteroatoms per ring and optionally substituted with one, two or three R⁵⁰ substituents;
   or R¹² and R¹³ together with the N to which they are covalently bound, a C₅-C₆ heterocycle optionally containing a second annular heteroatom and optionally substituted with one or two R⁵⁰ substituents; and

R⁵⁰ is R⁵¹-L³-(CH₂)ₙ—;
L³ is —O—, —NH—, —S(O)₀₋₂—, —C(O)—, —C(O)O—, —C(O)NH—, —OC(O)—, —NHC(O)—, —C₆H₄—, or a direct bond;
R⁵¹ is —H, C₁-C₆-alkyl, C₂-C₆-alkenyl, C₂-C₆-alkynyl, halo, —CF₃, —OH, —NH₂, mono-C₁-C₆alkyl amino, di-C₁-C₆alkyl amino, —SH, —CO₂H, —CN, —NO₂, —SO₃H, or a saturated or mono- or poly-unsaturated C₅-C₁₄-mono- or fused poly-cyclic hydrocarbyl, optionally containing one or two annular heteroatoms per ring and optionally substituted with one, two, or three substituents;

wherein n is 0, 1, 2, or 3;

provided that an O or S is not singly bonded to another O or S in a chain of atoms.

In embodiment 14, the invention comprises the method according to embodiment 13, wherein -L¹-R¹ is selected from Table 4,

TABLE 4

| | | |
|---|---|---|
| —R¹⁴ | 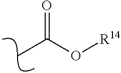 | 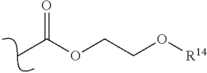 |
| 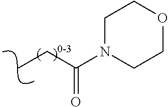 | 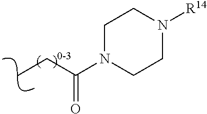 | 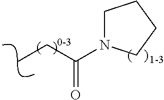 |
| 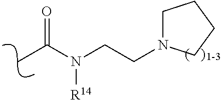 | 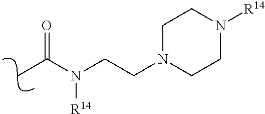 | 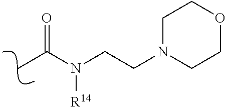 |
| 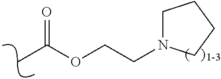 | 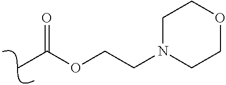 | 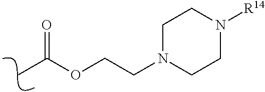 |
| 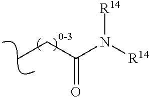 | 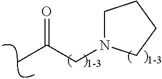 | 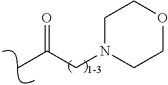 |
| 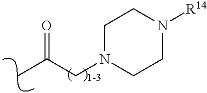 | 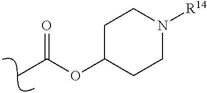 | 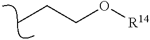 |
| 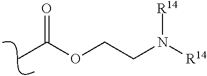 | 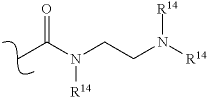 | 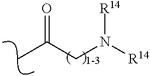 |
| 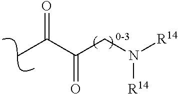 | 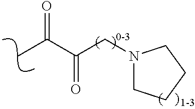 | 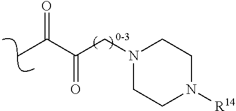 |
| 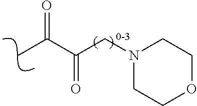 | 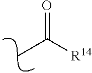 | 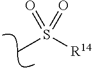 |
| 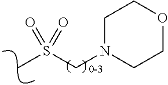 | 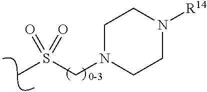 | 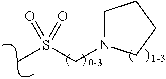 |
| 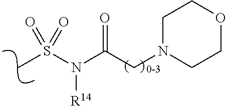 | 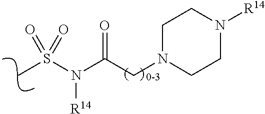 | 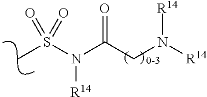 |
| 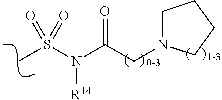 | 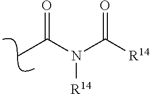 | 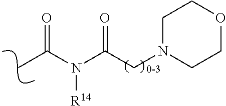 |
| 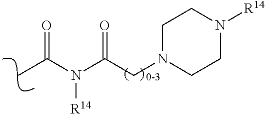 | 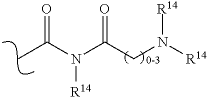 | 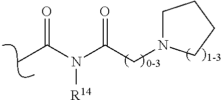 |

wherein each R¹⁴ is independently selected from —H, —(CH₂)₁₋₃CO₂H, alkyl, alkoxy, alkenyl, aryl, heteroaryl, arylalkyl, and heteroarylalkyl.

In embodiment 15, the invention comprises the method according to embodiment 14, wherein Z is —C(R¹⁵)═ or —C(H)═; L⁴ is —O—; and p is at least one.

In embodiment 16, the invention comprises the method according to embodiment 15, wherein Ar is selected from the group consisting of phenyl, biphenyl, napthyl, tetrahydronaphthalene, chromen-2-one, dibenzofuran, pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl, each optionally substituted.

In embodiment 17, the invention comprises the method according to embodiment 16, wherein Ar is phenyl, optionally substituted, with at least one halogen.

In embodiment 18, the invention comprises the method according to embodiment 17, wherein p is at least two.

In embodiment 19, the invention comprises the method according to embodiment 18, wherein -L¹-R¹ is —C(═O)OR¹⁴ or —(CH₂)₂OR¹⁴.

In embodiment 20, the invention comprises the method according to embodiment 19, wherein the compound has the structure:

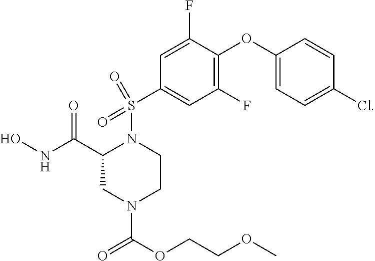

In embodiment 21, the invention comprises the method according to embodiment 14, wherein Z is —N═; and L⁴ is —O—.

In embodiment 22, the invention comprises the method according to embodiment 21, wherein Ar is selected from the group consisting of phenyl, biphenyl, napthyl, tetrahydronaphthalene, chromen-2-one, dibenzofuran, pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl, each optionally substituted.

In embodiment 23, the invention comprises the method according to embodiment 22, wherein Ar is optionally substituted tetrahydro-naphthalene.

In embodiment 24, the invention comprises the method according to embodiment 23, wherein -L¹-R¹ is —C(═O)OR¹⁴ or —(CH₂)₂₋₃OR¹⁴.

In embodiment 25, the invention comprises the method according to embodiment 24, wherein p is zero.

In embodiment 26, the invention comprises the method according to embodiment 25, having the structure:

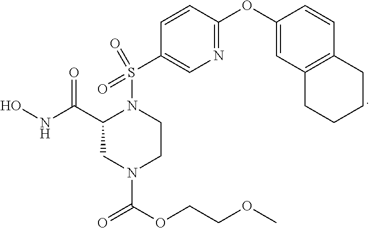

In embodiment 27, the invention comprises the method according to embodiment 14, wherein Z is —N═; and L⁴ is nothing.

In embodiment 28, the invention comprises the method according to embodiment 27, wherein Ar is selected from the group consisting of phenyl, biphenyl, napthyl, tetrahydronaphthalene, chromen-2-one, dibenzofuran, pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl, each optionally substituted.

In embodiment 29, the invention comprises the method according to embodiment 28, wherein p is zero.

In embodiment 30, the invention comprises the method according to embodiment 29, wherein Ar is optionally substituted phenyl.

In embodiment 31, the invention comprises the method according to embodiment 30, wherein -L¹-R¹ is —C(═O)OR¹⁴ or —(CH₂)₂₋₃OR¹⁴.

In embodiment 32, the invention comprises the method according to embodiment 31, having the structure:

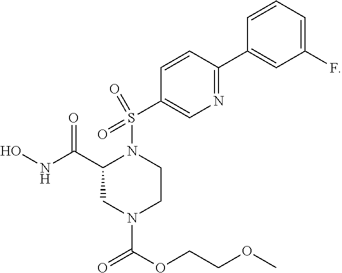

In embodiment 33, the invention comprises the method according to embodiment 14, of formula V,

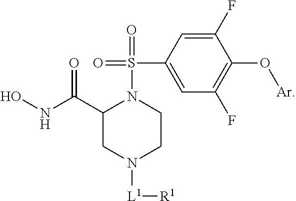

In embodiment 34, the invention comprises the method according to embodiment 33, wherein Ar is selected from the group consisting of phenyl, biphenyl, napthyl, tetrahydronaphthalene, chromen-2-one, dibenzofuran, pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl, each optionally substituted.

In embodiment 35, the invention comprises the method according to embodiment 34, wherein Ar is phenyl, optionally substituted, with at least one halogen.

In embodiment 36, the invention comprises the method according to embodiment 34, wherein Ar is selected from,

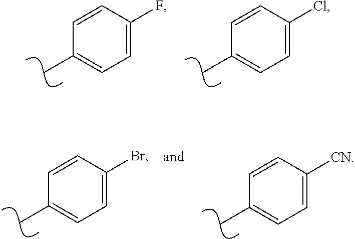

In embodiment 37, the invention comprises the method according to embodiment 35, wherein the absolute stereochemistry is according to formula VI,

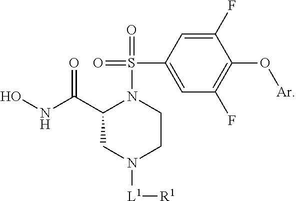

In embodiment 38, the invention comprises the method according to embodiment 37, wherein -L¹-R¹ is —C(═O)OR¹⁴ or —(CH₂)₂₋₃OR¹⁴.

In embodiment 39, the invention comprises the method according to embodiment 38, having the structure:

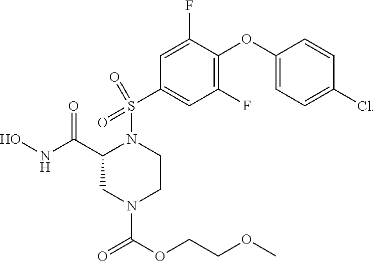

In embodiment 40, the invention comprises a method of treating aneurysmal dilatation or blood vessel wall weakness, including abdominal aortic aneurysm and thoracic aneurysm, comprising administering to a subject with an aneurysmal dilatation or blood vessel wall weakness, a therapeutically effective amount of a pharmaceutical composition comprising a compound as described in any of the embodiments 1-39 and a pharmaceutically acceptable carrier.

In embodiment 41, the invention comprises a method of treating aneurysmal dilatation or blood vessel wall weakness, including abdominal aortic aneurysm and thoracic aneurysm, comprising administering to a subject with an aneurysmal dilatation or blood vessel wall weakness a therapeutically effective amount of a sulfonyl halide according to formula VIII:

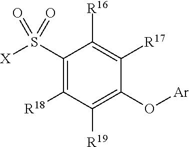

wherein X is halogen; R¹⁶, R¹⁷, R¹⁸, and R¹⁹, are each independently either —H or —F; and Ar is aryl or heteroaryl, each optionally substituted.

In embodiment 42, the invention comprises a method according to embodiment 41, wherein R¹⁶ and R¹⁸ are each —H; and R¹⁷ and R¹⁹ are each —F.

In embodiment 43, the invention comprises the method according to embodiment 42, wherein Ar is selected from the group consisting of phenyl, biphenyl, napthyl, tetrahydronaphthalene, chromen-2-one, dibenzofuran, pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl, each optionally substituted.

In embodiment 44, the invention comprises the method according to embodiment 43, wherein Ar is phenyl, optionally substituted, with at least one halogen.

In embodiment 45, the invention comprises the method according to embodiment 44, wherein the compound is of formula IX:

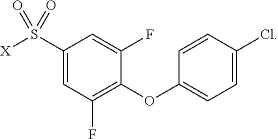

In embodiment 46, the invention comprises the method according to embodiment 45, wherein X is —Cl.

In embodiment 47, the invention comprises a method of treating aneurysmal dilatation or blood vessel wall weakness, including abdominal aortic aneurysm and thoracic aneurysm, comprising administering to a mammal in need of such treatment a therapeutically effective amount of a pharmaceutical composition according to embodiment 40.

In embodiment 48 of the invention is a method of modulating the activity of MMPs comprising administering to a mammal in need of such treatment a therapeutically effective amount of a pharmaceutical composition according to embodiment 40.

In embodiments 49-98, the invention comprises each of embodiments 1-49 wherein the recited compound is administered in combination with (simultaneously or serially) a therapeutically effective amount of an ACE inhibitor, an ARB, or cyclophilin inhibitor (e.g., cyclosporin A).

In embodiment 99, the invention comprises any one of the methods of embodiments 1-98, wherein the aneurysmal dilatation or the blood vessel wall weakness is an aortic aneurysm or a thoracic aneurysm.

In embodiment 100, the invention comprises a pharmaceutical composition comprising a compound as recited in any of embodiments 1-49, wherein the compound is present in an amount effective to treat an aneurysmal dilatation or a blood vessel wall weakness. In particular embodiments, the aneurysmal dilatation or blood vessel wall weakness is an abdominal aortic aneurysm or a thoracic aneurysm.

In embodiment 101, the invention comprises a pharmaceutical composition as described in embodiment 100, wherein the pharmaceutical composition further comprises a second therapeutic agent selected from ACE inhibitors, ARBs, or cyclophilin inhibitors, wherein the compound and the second therapeutic agent are present in an amount effective to treat an aneurysmal dilatation or a blood vessel wall weakness. In particular embodiments, the aneurysmal dilatation or blood vessel wall weakness is an abdominal aortic aneurysm or a thoracic aneurysm.

In embodiment 102, the invention comprises the pharmaceutical composition of embodiment 101, wherein the second therapeutic agent is an ACE inhibitor selected from captopril, zofenopril, enalapril, ramipril, quinapril, perindopril, lisinopril, benazepril, and fosinopril.

In embodiment 103, the invention comprises the pharmaceutical composition of embodiment 101, wherein the second therapeutic agent is an ARB selected from candesartan, aprosartan, irbesartan, valsartan, and losartan.

Many ACE inhibitors are known in the art. These include captopril, zofenopril, enalapril, ramipril, quinapril, perindopril, lisinopril, benazepril, and fosinopril.

Many ARBs are known in the art as well. For example, candesartan, aprosartan, irbesartan, valsartan, and losartan are currently available.

In view of the foregoing considerations, I recognized that effective therapies for aneurysmal dilatation or blood vessel wall weakness, including abdominal aortic aneurysms and thoracic aneurysms, are desirable.

The methods of the invention are expected to be effective because MMPs play an important role in tissue remodeling associated with various physiological and pathological processes, including angiogenesis, tissue repair, cirrhosis, arthritis, and metastasis. MMPs are also implicated in the breakdown of elastin and weakening of the aortic wall, resulting in aneurysmal dilatation, including abdominal aortic aneurysms and thoracic aneurysms. In view of the importance of MMPs in biological processes and disease states, inhibitors of these proteins are desirable, particularly small molecule inhibitors.

MMPs, excreted by immune and stromal cells, are known to cause medial degeneration, and increased plasma levels of MMPs have been correlated with the development and severity of peripheral artery disease. Furthermore, MMPs are thought to play a role in the degradation of extracellular matrix proteins that occurs during the development of aneurysms (see Sakalihasan et al, J Vasc Surg 1996; 24:127-33).

MMP inhibitors of the invention are expected to be useful for treating aneurysmal dilatation or blood vessel wall weakness, including abdominal aortic aneurysms and thoracic aneurysms, alone or in combination with other drugs. With respect to abdominal aortic aneurysms, studies have suggested that, in addition to MMPs, other proteins may play a role in aneurysm formation, including angiotensin II and cyclophilins. Therefore, the combination therapies of the invention are expected to be effective in targeting multiple aspects of the disease process. I recognized that therapies that combine inhibitors of matrix metalloproteases and inhibitors of angiotensin II and cyclophilins may prove to be more effective than these therapies individually.

I expect that combination therapies of embodiments 49-98 will be particularly effective at treating aneurysmal dilatation or blood vessel wall weakness because combination therapies will allow for the targeting of multiple aspects of the disease processes; MMPs, angiontensin II, and cyclophilin have all been implicated in these diseases. Cyclophilin A, for instance, binds to CD147, which is a known inducer of extracellular matrix metalloproteinase. This binding causes CD147 to translocate to the cell surface where it plays a critical role in stimulating matrix metalloproteinase activity, thereby leading to matrix degradation that results in abdominal aortic aneurysm. By inhibiting cyclophilin A, it is thought that matrix degradation can be reduced. Additionally, angiotensin II appears to cause the release of cyclophilin A, which induces matrix metalloproteinase-2. Inhibition of angiotensin II is therefore thought to inhibit matrix metalloproteinase, thereby reducing matrix degradation.

Compounds disclosed herein were previously identified as ADAM-10 inhibitors (U.S. Publication No. 20060199820).

### DEFINITIONS

The following paragraphs provide definitions of the various chemical moieties that make up the compounds of the invention and are intended to apply uniformly throughout the specification and claims unless expressly stated otherwise.

The term alkyl refers inclusively to a univalent C₁ to C₂₀ (unless explicitly stated otherwise) saturated straight, branched, cyclic, and combinations thereof alkane moiety and specifically includes methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. In certain instances, specific cycloalkyls are defined (e.g. C₃-C₈ cycloalkyl) to differentiate them from generically described alkyls (that, again, are intended to construe inclusion of cycloalkyls). Thus “alkyl” includes, e.g., C₃-C₈ cycloalkyl. The term “alkyl” also includes, e.g., C₃-C₈ cycloalkyl C₁-C₆ alkyl, which is a C₁-C₆ alkyl having a C₃-C₈ cycloalkyl terminus. Alkyl's can be optionally substituted with any appropriate group, including but not limited to one or more moieties selected from halo, hydroxyl, amino, arylalkyl, heteroarylalkyl, alkylamino, arylamino, alkoxy, aryloxy, nitro, cyano, sulfonic acid, sulfate, phosphonic acid, phosphate, or phosphonate, either unprotected, or protected as necessary, as known to those skilled in the art or as taught, for example, in Greene, et al., “Protective Groups in Organic Synthesis,” John Wiley and Sons, Second Edition, 1991.

The term alkoxy refers to the group —O-(substituted alkyl), the substitution on the alkyl group generally containing more than only carbon (as defined by alkoxy). One exemplary substituted alkoxy group is “polyalkoxy” or —O— (optionally substituted alkylene)-(optionally substituted alkoxy), and includes groups such as —OCH₂CH₂OCH₃, and glycol ethers such as polyethyleneglycol and —O(CH₂CH₂O)ₓCH₃, where x is an integer of between about 2 and about 20, in another example, between about 2 and about 10, and in a further example between about 2 and about 5. Another exemplary substituted alkoxy group is hydroxyalkoxy or —OCH₂(CH₂)_{y}OH, where y is for example an integer of between about 1 and about 10, in another example y is an integer of between about 1 and about 4.

The term alkenyl refers to a univalent C₂-C₆ straight, branched, or in the case of C₅₋₈, cyclic hydrocarbon with at least one double bond.

The term aryl refers to a univalent phenyl, biphenyl, napthyl, and the like. The aryl group can be optionally substituted with any suitable group, including but not limited to one or more moieties selected from halo, hydroxyl, amino, alkylamino, arylamino, alkoxy, aryloxy, nitro, cyano, sulfonic acid, sulfate, phosphoric acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991). As well, substitution on an aryl can include fused rings such as in tetrahydronaphthalene, chromen-2-one, dibenzofuran, and the like. In such cases, e.g. tetrahydronaphthalene, the aryl portion of the tetrahydronaphthalene is attached to the portion of a molecule described as having an aryl group.

The term heteroatom means O, S, P, or N.

The term heterocycle refers to a cyclic alkyl, alkenyl, or aryl moiety as defined above wherein one or more ring carbon atoms is replaced with a heteroatom. A heterocycle also refers to a fused bi- or tri-cyclic moiety in which one ring is s aromatic and one ring is not and one of the rings contains an annular heteroatom.

The term heteroaryl specifically refers to an aryl that includes at least one of sulfur, oxygen, and nitrogen in the aromatic ring. Non-limiting examples are pyryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, benzothienyl, isobenzofuryl, pyrazolyl, indolyl, purinyl, carbazolyl, benzimidazolyl, and isoxazolyl.

The term halo refers to chloro, fluoro, iodo, or bromo.

As used herein, the term pharmaceutically acceptable salts or complexes refers to salts or complexes that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenyl-acetate).

The term “pharmaceutically active derivative” refers to any compound that, upon administration to the recipient, is capable of providing directly or indirectly, the compounds disclosed herein.

In some examples, as will be appreciated by those skilled in the art, two adjacent carbon containing groups on an aromatic system may be fused together to form a ring structure. The fused ring structure may contain heteroatoms and may be substituted with one or more substitution groups “R”. It should additionally be noted that for cycloalkyl (i.e. saturated ring structures), each positional carbon may contain two substitution groups, e.g. R and R′.

Some of the compounds of the invention may have imino, amino, oxo or hydroxy substituents off aromatic heterocyclic ring systems. For purposes of this disclosure, it is understood that such imino, amino, oxo or hydroxy substituents may exist in their corresponding tautomeric form, i.e., amino, imino, hydroxy or oxo, respectively.

Compounds of the invention are generally named using ACD/Name (available from Advanced Chemistry Development, Inc. of Toronto, Canada). This software derives names from chemical structures according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. When desired, the R- and S-isomers may be resolved by methods known to one skilled in the art, for example by: formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer may be further enriched (with concomitant loss in yield) by recrystallization.

“Optional” or “optionally” means that the subsequently described event or circumstance may or may not occur, and that the description includes instances where said event or circumstance occurs and instances in which it does not. It will be understood by one skilled in the art with respect to any group containing one or more substituents that such groups are not intended to introduce any substitution or substitution patterns that are sterically impractical and/or synthetically non-feasible. “Optionally substituted” refers to all subsequent modifiers in a term, for example in the term “optionally substituted C₁₋₈alkylaryl,” optional substitution may occur on both the “C₁₋₈alkyl” portion and the “aryl” portion of the molecule; and for example, optionally substituted alkyl includes optionally substituted cycloalkyl groups, which in turn are defined as including optionally substituted alkyl groups, potentially ad infinitum.

“Substituted” alkyl, aryl, and heterocyclyl, for example, refer respectively to alkyl, aryl, and heterocyclyl, wherein one or more (for example up to about 5, in another example, up to about 3) hydrogen atoms are replaced by a substituent independently selected from, but not limited to: optionally substituted alkyl (e.g., fluoroalkyl), optionally substituted alkoxy, alkylenedioxy (e.g. methylenedioxy), optionally substituted amino (e.g., alkylamino and dialkylamino), optionally substituted amidino, optionally substituted aryl (e.g., phenyl), optionally substituted arylalkyl (e.g., benzyl), optionally substituted aryloxy (e.g., phenoxy), optionally substituted arylalkyloxy (e.g., benzyloxy), carboxy (—COOH), carboalkoxy (i.e., acyloxy or —OOCR), carboxyalkyl (i.e., esters or —COOR), carboxamido, aminocarbonyl, benzyloxycarbonylamino (CBZ-amino), cyano, carbonyl, halogen, hydroxy, optionally substituted heterocyclylalkyl, optionally substituted heterocyclyl, nitro, sulfanyl, sulfinyl, sulfonyl, and thio.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about 1 and about 6 carbons) wherein the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about 1 and about 6 carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

“Metabolite” refers to the break-down or end product of a compound or its salt produced by metabolism or biotransformation in the animal or human body; e.g., biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be synthesized such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Therapeutically effective amount” refers to the amount of agent that has a beneficial effect, which may be curative or palliative, on the health and well-being of a patient with regard to a disease with which the patient is known or suspected to be afflicted. A therapeutically effective amount may be administered as a single bolus, as intermittent bolus charges, as short, medium or long term sustained release formulations or as any combination of these.

“Treatment,” “method of treatment,” and “treating” refer to the administration of a therapeutically effective amount of an agent to a patient known or suspected to be suffering from a disease, including aneurysmal dilatation or blood vessel wall weakness, for example abdominal aortic aneurysm and thoracic aneurysm. Such treatment may be curative or palliative. Agents useful with this invention are described herein.

A therapeutic “agent” refers to a bioactive agent that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to regress; or (4) alleviating one or more symptoms of the disease.

A bioactive agent also refers to an agent that, when administered to a patient, either prevents the occurrence of a disease or disorder or retards the recurrence of the disease or disorder. Such a bioactive agent is often referred to as a prophylactic bioactive agent.

“Mammal” refers to a mammalian patient, including but not limited to a human patient.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

### Experimental Section

The compounds of the invention can be made in accordance with the following general description and following the teachings provided in the Example Section, below, and methods routine to those of ordinary skill in the art. The examples are merely illustrative and are not intended to be limiting.

N-Hydroxy-1,4-disubstituted piperazine-2-carboxamides of the present invention can be synthesized using the methods described below. Method A begins with the reaction of piperazine-2-(R)-carboxylic acid dihydrochloride (1), for example, with di-tert-butyl dicarbonate to yield the bis-Boc protected intermediate 2, which is esterified, for example, with methyl iodide in the presence of cesium carbonate to form methyl ester 3. The Boc groups are then removed from 3 to yield piperazine dihydrochloride intermediate 4.

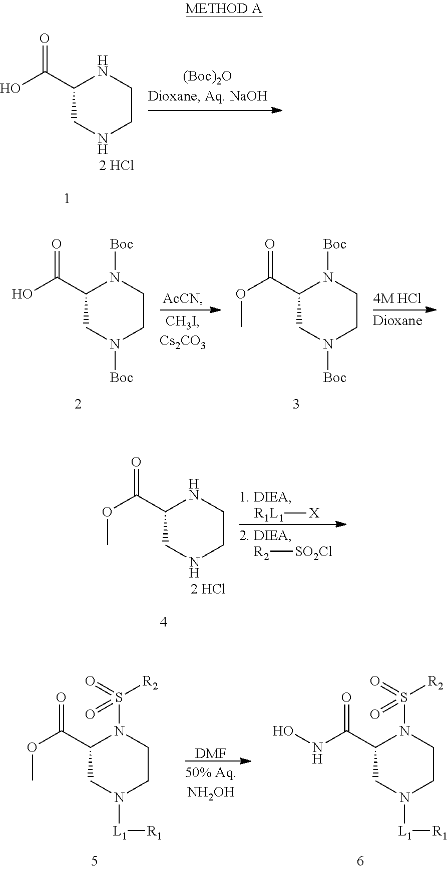

In one pot, the N4 nitrogen of 4 is selectively acylated, carbamylated, sulfonylated, alkylated, and the like, followed by sulfonylation of the N1 nitrogen to faun the disubstituted piperazine 5. The methyl ester group of 5 is then converted to the hydroxamate in a mixture of DMF and 50% aqueous hydroxylamine, for example, to give the corresponding N-hydroxy-1,4-disubstituted piperazine-2-(R)-carboxamide 6, in accordance with formula I.

Method B begins with the sulfonylation of the N1 nitrogen of mono-Boc protected piperazine-2-(R)-carboxylic acid 7, for example, through the use of trimethylsilyl chloride and an appropriate sulfonyl chloride (see synthesis below) to form intermediate 8. Intermediate 8 is then esterified with TMS-diazomethane to form methyl ester 9, followed by deprotection of the Boc group with TFA to form the TFA salt of 10. Alternatively, compound 8 can be simultaneously esterified and Boc-deprotected using HCl in methanol to form the HCl salt of 10. The N4 nitrogen of 10 is acylated, carbamylated, sulfonylated, alkylated, etc. to form methyl ester 5, which is converted to the hydroxamate 6 (see structure in Method A description) using a mixture of DMF and 50% aqueous hydroxylamine as described above or, alternatively, by treatment with hydroxylamine under basic conditions (KOH in MeOH).

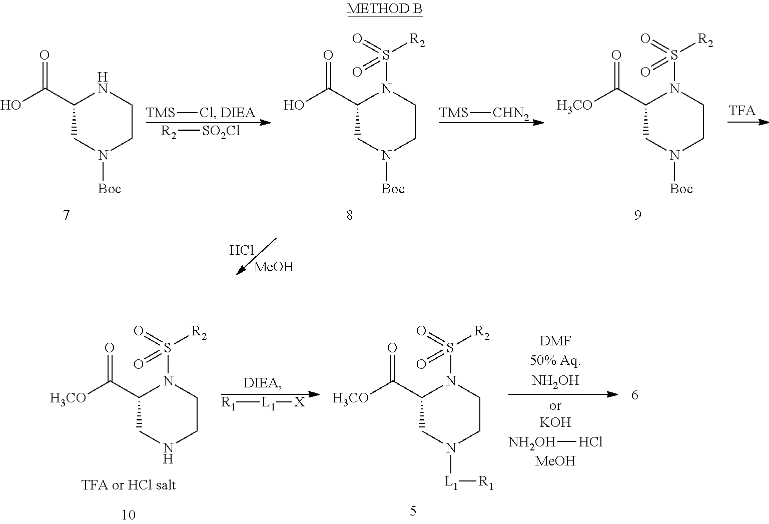

Method C begins with the one pot synthesis of the disubstituted piperazine-2-(R)-carboxylic acid 8 from the dihydrochloride 1. First, under Schotten-Baumann conditions, the N4 nitrogen of 1 is selectively Boc-protected, followed by the addition of triethylamine and the appropriate sulfonyl chloride to sulfonylate the N1 nitrogen to form 8. From intermediate 8, the desired hydroxamates 6 are formed as described in Method B.

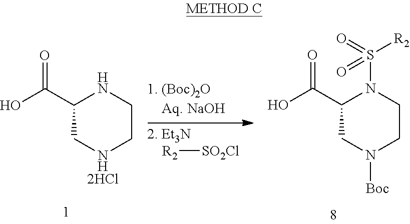

### EXAMPLE SECTION

### Example 1

### N-Hydroxy-1-[4-(4-fluorophenoxy)-phenyl)]sulfonyl-4-(4-morpholinyl-carbonyl)piperazine-2-(R)-carboxamide (Method A)

### Step 1—Formation of 1,4-di-tert-butoxycarbonyl-piperazine-2-(R)-carboxylic acid

Piperazine-2-(R)-carboxylic acid dihydrochloride (16.6 g, 82 mmol) and dioxane (120 ml) were combined and cooled in an icebath. 5N NaOH (60 ml, 300 mmol) was added, followed by (Boc)₂O (41.8 g, 191 mmol). The reaction mixture was allowed to warm to room temperature with stirring over several hours, then concentrated in vacuo. The resulting aqueous mixture was washed with Et₂O (3×), cooled in an icebath, acidified to pH 2-3 with concentrated HCl and extracted with EtOAc (3×). Combined EtOAc extractions were washed with water (1×), saturated NaCl (1×), dried (Na₂SO₄), and concentrated in vacuo to give 1,4-di-tert-butoxycarbonylpiperazine-2-(R)-carboxylic acid as a white solid (27.0 g, 100%). LC/MS Calcd for [M−H]⁻ 329.2, found 329.2.

### Step 2—Formation of methyl 1,4-di-tert-butoxycarbonyl piperazine-2-(R)-carboxylate

1,4-Di-tert-butoxycarbonylpiperazine-2-(R)-carboxylic acid (70 g, 212 mmol) was dissolved in acetonitrile (1.3 L). Cs₂CO₃ (110 g, 340 mmol) was added and the mixture stirred for 30 minutes at room temperature before the addition of methyl iodide (28 ml, 450 mmol). The reaction mixture was stirred at room temperature overnight, solids were filtered and the filtrate concentrated in vacuo. The resulting oil was dissolved in EtOAc and any insoluble material filtered. The filtrate was concentrated in vacuo to give methyl 1,4-di-tert-butoxycarbonylpiperazine-2-(R)-carboxylate (69 g, 95%). LC/MS Calcd for [M+H]⁺ 345.2, found 145.1 (-Boc X 2).

### Step 3—Formation of methyl piperazine-2-(R)-carboxylate dihydrochloride

Methyl 1,4-di-tert-butoxycarbonylpiperazine-2-(R)-carboxylate (2.9 g, 8.5 mmol) was dissolved in 4M HCl in dioxane (30 ml) and stirred at room temperature for 30-60 minutes, farming a thick white precipitate. The reaction mixture was concentrated in vacuo and the resulting white solid dried under high vacuum to give methyl piperazine-2-(R)-carboxylate dihydrochloride (1.9 g, 100%). LC/MS Calcd for [M+H]⁺ 145.1, found 145.1.

### Step 4—Formation of methyl 1-[4-(4-fluoro-phenoxy)phenyl)]sulfonyl-4-(4-morpholinylcarbonyl)pipera-zine-2-(R)-carboxylate

Methyl piperazine-2-(R)-carboxylate dihydrochloride (676 mgs, 3.1 mmol) was dissolved in CH₂Cl₂ (7 mls) and DIEA (2.1 mls, 12.4 mmol) and cooled in an icebath. Morpholinecarbonyl chloride (450 mgs, 3.0 mmol) dissolved in methylene chloride (2.5 mls) was added dropwise with stirring. After addition was complete, the reaction mixture was allowed to warm to room temperature and stirred for an additional 2-3 hrs. Additional DIEA (0.6 mls, 3.4 mmol) was added, followed by 4-(4-fluorophenoxy)phenylsulfonyl chloride (904 mg, 3.1 mmol) and the reaction mixture stirred at room temperature overnight. The reaction mixture was concentrated in vacuo and the resulting residue redissolved in EtOAc and washed with water (1×), 1.0N HCl (2×), dried (Na₂SO₄), concentrated in vacuo and purified by flash chromatography (3:1 EtOAc:hexanes) to give methyl 1-[4-(4-fluorophenoxy)phenyl)]sulfonyl-4-(4-morpholinylcarbonyl)piperazine-2-(R)-carboxylate (1.11 g, 70%). LC/MS Calcd for [M+H]⁺ 508.1, found 508.1.

### Step 5—Formation of N-hydroxy-1-[4-(4-fluorophenoxy)phenyl)]sulfonyl-4-(4-morpholinylcarbonyl)piperazine-2-(R)-carbox-amide

Methyl 1-[4-(4-fluorophenoxy)phenyl)]sulfonyl-4-(4-morpholinylcarbonyl)piperazine-2-(R)-carboxylate (1.11 g, 2.2 mmol) was dissolved in DMF (17 mls) to which was added 50% aqueous NH₂OH (20 mls) and the reaction mixture stirred at room temperature overnight. The reaction mixture was poured into cold 1.0N HCl (100-120 mls) and extracted with EtOAc (4×). The combined EtOAc extractions were washed with 10% aqueous LiCl (4×), saturated NaCl (1×), dried (Na₂SO₄), and concentrated in vacuo. The crude product was purified by flash chromatography (EtOAc) and the resulting pure oil was dissolved in 1:1 acetonitrile:water and lyophilized to give N-hydroxy-1-[4-(4-fluorophenoxy)phenyl)]sulfonyl-4-(4-morpholinyl-carbonyl)piperazine-2-(R)-carboxamide as a white solid (659 mg, 59%). LC/MS Calcd for [M+H]⁺ 509.1, found 509.1. ¹HNMR (400 MHz, CD₃OD): δ 7.69 (d, 2H, J=9.2 Hz), 7.04 (m, 4H), 6.95 (d, 2H, J=9.2 Hz), 4.30 (m, 1H), 3.76 (m, 1H), 3.50 (m, 7H), 3.10 (m, 4H), 2.90 (dd, 1H, J=13.2, 4.4 Hz), 2.72 (m, 1H).

### Example 2

### N-Hydroxy-1-[4-(4-fluorophenoxy)-3,5-difluorophenyl)]sulfonyl-4-(ethoxycarbonyl)piperazine-2-(R)-carboxamide (Method B)

### Step 1—Formation of 1-[4-(4-fluorophenoxy)-3,5-difluoro-phenyl)]sulfonyl-4-boc-piperazine-2-(R)-carboxylic acid

4-Boc-piperazine-2-(R)-carboxylic acid (933 mg, 4.05 mmol), CH₂Cl₂ (12 ml), DMF (6 ml), and DIEA (2.5 ml, 14.3 mmol) were combined under N₂. TMS-Cl (810 μl, 6.38 mmol) was added slowly and the mixture stirred at room temperature for approximately 2 hrs. 4-(4-fluorophenoxy)-3,5-difluorophenyl)]sulfonyl chloride (1.43 g, 4.43 mmol) dissolved in a minimum of CH₂Cl₂ was added and the mixture stirred at room temperature for another 2 hrs. The reaction mixture was diluted with EtOAc and washed with 0.5N HCl (3×), sat'd NaCl (1×), dried (Na₂SO₄), and concentrated in vacuo. The resulting crude oil was purified by flash chromatography (6:4 hexanes:EtOAc+1% AcOH) to give the desired product (1.37 g, 65%). LC/MS Calcd for [M+H]⁺ 517.1, found 417.0 (-Boc).

### Step 2—Formation of methyl 1-[4-(4-fluorophenoxy)-3,5-difluorophenyl)]sulfonyl-4-boc-piperazine-2-(R)-carboxylate

1-[4-(4-fluorophenoxy)-3,5-difluorophenyl)]sulfonyl-4-boc-piperazine-2-(R)-carboxylic acid (1.37 g, 2.65 mmol) was dissolved in CH₂Cl₂ (40 ml) and MeOH (10 ml). A mixture of 2M TMS-CHN₂ in hexanes (2.5 ml, 5 mmol) and CH₂Cl₂ (10 ml) was added dropwise with stirring and the reaction followed by TLC. Upon completion of the reaction, AcOH (1.0 ml) was added dropwise with stirring. The reaction mixture was further diluted with CH₂Cl₂ and washed with water (1×), saturated NaHCO₃ (2×), saturated NaCl (1×), dried (MgSO₄), and concentrated in vacuo. The crude oil was purified by flash chromatography (3:1 hexanes:EtOAc) to give the desired product (1.10 g, 78%). LC/MS Calcd for [M+H]⁺ 531.1, found 431.0 (-Boc).

### Step 3—Formation of methyl 1-[4-(4-fluorophenoxy)-3,5-difluorophenyl)]sulfonyl-piperazine-2-(R)-carboxylate TFA salt

Methyl 1-[4-(4-fluorophenoxy)-3,5-difluorophenyl)]sulfonyl-4-boc-piperazine-2-(R)-carboxylate (1.10 g, 2.07 mmol) was dissolved in a minimum of CH₂Cl₂ to which was added neat TFA (10 ml). The mixture was stirred at room temperature for approximately 30 min, concentrated in vacuo, further dried for several hours under high vacuum and used without further purification. LC/MS Calcd for [M+H]⁺ 431.1, found 431.0.

### Step 4—Formation of methyl 1-[4-(4-fluorophenoxy)-3,5-difluorophenyl)]sulfonyl-4-(ethoxycarbonyl)piperazine-2-(R)-carboxylate

To a mixture of methyl 1-[4-(4-fluorophenoxy)-3,5-difluorophenyl)]sulfonyl-piperazine-2-(R)-carboxylate TFA salt (344 mg, 0.63 mmol), CH₂Cl₂ (10 ml), and DIEA (250 μl, 1.43 mmol) under N₂ was added ethyl chloroformate (65 μl, 0.68 mmol). The mixture was stirred under N₂ at room temperature for 1.5 hrs, then washed with 1.0N HCl (2×), saturated NaCl (1×), dried (Na₂SO₄), and concentrated in vacuo. The crude residue was purified by flash chromatography (3:1 hexanes:EtOAc) to give the desired product (218 mgs, 69%). LC/MS Calcd for [M+H]⁺ 503.1, found 503.0.

### Step 5—Formation of N-hydroxy-1-[4-(4-fluorophenoxy)-3,5-difluorophenyl)]sulfonyl-4-(ethoxycarbonyl)piperazine-2-(R)-carboxamide

A 1.7M solution of NH₂OH in MeOH was prepared by mixing a solution of KOH (2.80 g, 50.0 mmol) in MeOH (7.0 ml) with a hot solution of NH₂OH HCl salt (2.40 g, 34.5 mmol) in MeOH (12.0 ml) and filtering the resulting solids after cooling to room temperature. Methyl 1-[4-(4-fluorophenoxy)-3,5-difluorophenyl)]-sulfonyl-4-(ethoxycarbonyl)piperazine-2-(R)-carboxylate (218 mg, 0.43 mmol) was dissolved in the 1.7M NH₂OH in MeOH solution (4.0 ml) and stirred at room temperature for 30-45 minutes. The reaction mixture was then diluted with 1.0N HCl and extracted with EtOAc (3×). Combined EtOAc extractions were washed with saturated NaCl (1×), dried (Na₂SO₄), and concentrated in vacuo. The resulting crude residue was purified by flash chromatography (1:1 EtOAc:hexanes) to give a colorless film which was lyophilized from 1:1 AcCN:H₂O to give the desired product as a white solid (136 mg, 62%). LC/MS Calcd for [M+H]⁺ 504.1, found 504.0. ¹HNMR (400 MHz, CD₃OD): δ 7.58 (m, 2H), 7.03 (m, 4H), 4.27 (m, 2H), 4.07 (m, 3H), 3.75 (m, 2H), 3.30 (m, 1H), 3.06 (m, 1H), 1.22 (m, 3H).

### Example 3

### N-Hydroxy-1-[4-(4-cyanophenoxy)-3-fluorophenyl)]sulfonyl-4-(2-methoxy-1-ethoxycarbonyl)piperazine-2-(R)-carboxamide

### (Method C)

### Step 1—Formation of 1-[4-(4-cyanophenoxy)-3-fluorophenyl)]sulfonyl-4-boc-piperazine-2-(R)-carboxylic acid

Piperazine-2-(R)-carboxylic acid dihydrochloride (1.25 g, 6.1 mmol), dioxane (15 mls) and water (6.0 mls) were combined and cooled in an icebath. 9N NaOH (2.0 mls, 18 mmol) was added slowly with stirring, followed by (Boc)₂O (1.35 g, 6.2 mmol). The reaction mixture was allowed to warm to room temperature and stirred for an additional 3-4 hrs. Et₃N (1.8 mls, 13 mmol) was added, followed by 4-cyanophenoxy-3-fluorophenylsulfonyl chloride (2.00 g, 6.4 mmol). The reaction mixture is stirred at room temperature for 1-2 hrs, then concentrated in vacuo. The resulting residue was partitioned between 1.0N HCl and EtOAc. Phases were separated and the aqueous phase was further extracted with EtOAc (2×). Combined EtOAc extractions were washed with 1.0N HCl (1×), saturated NaCl (1×), dried (MgSO₄), and concentrated in vacuo. The resulting residue is purified by flash chromatography (7:3 hexanes:EtOAc+1% AcOH) to give the desired product (1.1 g, 35%). LC/MS Calcd for [M−H]⁻ 504.1, found 504.3.

### Step 2

Methyl 1-[4-(4-cyanophenoxy)-3-fluorophenyl)]sulfonyl-4-boc-piperazine-2-(R)-carboxylate was made in the same manner as Example 2, step 2, except purification by flash chromotography was unnecessary. 1.10 g recovered (97%). LC/MS Calcd for [M+H]⁺ 520.1, found 420.1 (-Boc).

### Step 3

Methyl 1-[4-(4-cyanophenoxy)-3-fluorophenyl)]sulfonyl-piperazine-2-(R)-carboxylate TFA salt was made in the same manner as Example 2, step 3. LC/MS Calcd for [M+H]⁺ 420.1, found 420.2.

### Step 4

Methyl 1-[4-(4-cyanophenoxy)-3-fluorophenyl)]sulfonyl-4-(2-methoxy-1-ethoxycarbonyl)piperazine-2-(R)-carboxylate was made in the same manner as Example 2, step 4. 438 mgs recovered (83%). LC/MS Calcd for [M+H]⁺ 522.1, found 522.2.

### Step 5

N-Hydroxy-1-[4-(4-cyanophenoxy)-3-fluorophenyl)]sulfonyl-4-(2-methoxy-1-ethoxycarbonyl)piperazine-2-(R)-carboxamide was made in the same manner as Example 2, step 5. 46 mg recovered (10%). LC/MS Calcd for [M−H]⁻ 521.1, found 521.2. ¹HNMR (400 MHz, CD₃OD): δ 7.73 (m, 3H), 7.65 (m, 1H), 7.34 (m, 1H), 7.19 (d, 2H, J=8.4 Hz), 4.29 (m, 2H), 4.14 (m, 3H), 3.74 (m, 2H), 3.55 (m, 2H), 3.33 (s, 3H), 3.25 (m, 1H), 3.04 (m, 1H).

### Example 4

### Synthesis of Sulfonyl Chloride Intermediates

### Example 4a

### 4-(4-fluorophenoxy)-3,5-difluorophenylsulfonyl chloride

### Step 1

A mixture of 3,4,5-trifluoronitrobenzene (20.0 g, 113 mmol, commercially available from AsymChem of Durham, N.C.), dry DMF (100 ml), 4-fluorophenol (13.9 g, 124 mmol), and Cs₂CO₃ (56 g, 172 mmol) was stirred under N₂ at 60-70° C. for 1-2 hrs. After cooling to room temperature, the reaction mixture was partitioned between H₂O and EtOAc. The phases were separated and the aqueous phase was further extracted with EtOAc (2×). The EtOAc extractions were washed with sat'd NaCl (1×), dried over Na₂SO₄, and concentrated in vacuo to give 4-(4-fluorophenoxy)-3,5-difluoronitrobenzene (32.0 g, 105%) which was used in the next step without further purification. ¹H NMR (DMSO-d₆): δ 7.15 (m, 2H), 7.22 (m, 2H), 8.31 (d, 2H, J=7.6 Hz).

### Step 2

A mixture of 4-(4-fluorophenoxy)-3,5-difluoro-nitrobenzene (30.4 g, 113 mmol), EtOAc (300 ml), 10% Pd/C (2.6 g) was stirred under an atmosphere of H₂ at room temperature and pressure for approximately 6 hrs. The reaction mixture was filtered through Celite and concentrated in vacuo to give 4-(4-fluorophenoxy)-3,5-difluoroaniline (26.5 g, 98%) which was used in the next step without further purification. ¹H NMR (CDCl₃): δ 3.82 (s, 2H), 6.26 (d, 2H, J=8.4 Hz), 6.88 (m, 2H), 6.93 (m, 2H).

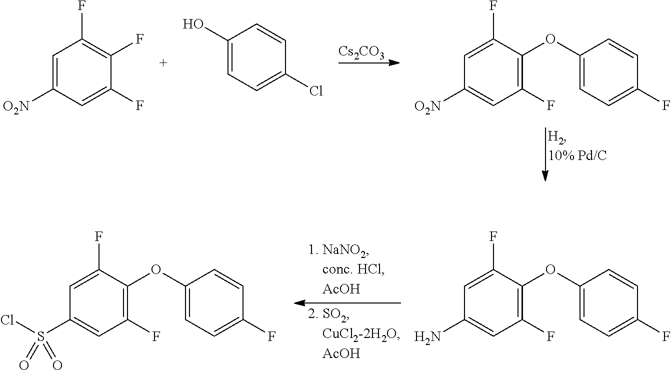

### Step 3

A solution of NaNO₂ (8.4 g, 122 mmol) in H₂O (20 ml) was added dropwise to a mixture of 4-(4-fluorophenoxy)-3,5-difluoroaniline (26.5 g, 111 mmol), AcOH (160 ml), and conc. HCl (160 ml) cooled in an ice/NaCl/H₂O bath. After addition was complete, the mixture was stirred an additional 20-30 minutes before a mixture of SO₂ (74 g, 1.15 mol) in AcOH (140 ml) and CuCl₂-2H₂O (11.1 g, 65 mmol) in H₂O (16 ml) was added. The reaction mixture was removed from the ice bath and stirred at room temperature for 1-2 hrs. The reaction mixture was poured into ice water and extracted with CH₂Cl₂ (3×). The combined CH₂Cl₂ extractions were washed with sat'd NaCl (1×), dried over Na₂SO₄, and concentrated in vacuo. The resulting crude oil was purified by flash chromatography (9:1 hexanes:EtOAC) to give 4-(4-fluorophenoxy)-3,5-difluorophenyl sulfonyl chloride (29.8 g, 83%). ¹H NMR (CDCl₃): δ 6.94 (m, 2H), 7.10 (m, 2H), 7.71 (d, 2H, J=6.4 Hz).

### Example 4b

### 4-(4-Chlorophenoxy)-3,5-difluorophenylsulfonyl chloride

### Step 1

A mixture of 3,4,5-trifluoronitrobenzene (6.6 g, 37 mmol), dry DMF (30 ml), 4-chlorophenol (5.26 g, 41 mmol), and Cs₂CO₃ (18.8 g, 58 mmol) was stirred under N₂ at 60-70 C for 1-2 hrs. After cooling to room temperature, the reaction mixture was partitioned between H₂O and EtOAc. The phases were separated and the aqueous phase was further extracted with EtOAc (2×). The EtOAc extractions were washed with sat'd NaCl (1×), dried over Na₂SO₄, and concentrated in vacuo to give 4-(4-chlorophenoxy)-3,5-difluoronitrobenzene (11.3 g, 106%) which was used in the next step without further purification. ¹H NMR (CDCl₃): δ 6.90 (d, 2H, J=7.6 Hz), 7.28 (d, 2H, J=7.6 Hz), 7.94 (d, 2H, J=6.4 Hz). Note: K₂CO₃/acetonitrile can be used in lieu of Cs₂CO₃/DMF.

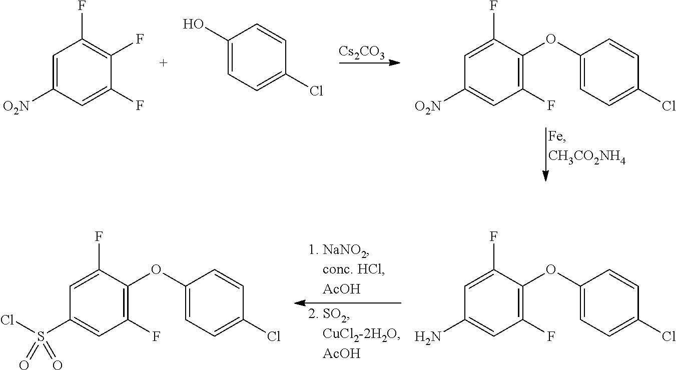

### Step 2

A mixture of 4-(4-chlorophenoxy)-3,5-difluoronitrobenzene (10.6 g, 37 mmol), toluene (150 ml), H₂O (150 ml), iron powder (6.9 g, 124 mmol), and ammonium acetate (9.3 g, 120 mmol) was heated to reflux with stirring for 2-3 hrs. After cooling to room temperature, the reaction mixture was filtered through Celite with thorough washing with H₂O and EtOAc. The filtrate was transferred to a separatory funnel and the phases separated. The aqueous phase was further extracted with EtOAc (2×). The combined organic phases were washed with H₂O (1×), sat'd NaCl (1×), dried over Na₂SO₄, and concentrated in vacuo to give 4-(4-chlorophenoxy)-3,5-difluoroaniline (10.8 g, 113%) which was used in the next step without further purification. ¹H NMR (CDCl₃): δ 3.81 (s, 2H), 6.27 (d, 2H, J=9.2 Hz), 6.85 (d, 2H, J=9.2 Hz), 7.21 (d, 2H, J=9.2 Hz).

### Step 3

A solution of NaNO₂ (2.8 g, 41 mmol) in H₂O (7.0 ml) was added dropwise to a mixture of 4-(4-chlorophenoxy)-3,5-difluoroaniline (9.5 g, 37 mmol), AcOH (50 ml), and conc. HCl (50 ml) cooled in an ice/NaCl/H₂O bath. After addition was complete, the mixture was stirred an additional 20-30 minutes before a mixture of SO₂ (25 g, 290 mmol) in AcOH (50 ml) and CuCl₂-2H₂O (3.8 g, 22 mmol) in H₂O (6.0 ml) was added. The reaction mixture was removed from the ice bath and stirred at room temperature for 1-2 hrs. The reaction mixture was poured into ice water and extracted with CH₂Cl₂ (3×). The combined CH₂Cl₂ extractions were washed with sat'd NaCl (1×), dried over Na₂SO₄, and concentrated in vacuo. The resulting crude oil was purified by flash chromatography (9:1 hexanes:EtOAC) to give 4-(4-chlorophenoxy)-3,5-difluorophenylsulfonyl chloride (11.0 g, 87%). ¹H NMR (CDCl₃): δ 6.92 (d, 2H, J=7.2 Hz), 7.30 (d, 2H, J=7.2 Hz), 7.72 (d, 2H, J=4.8 Hz).

### Example 4c

### 3,4,5-trifluorobenzenesulfonyl chloride

To a 2000 mL round-bottomed flask was added 800 mL distilled H₂O and a stir bar. Upon stirring, the flask was cooled to −10° C. in an ice-acetone bath. The flask was fitted with a 500 mL addition funnel and SOCl₂ (300 mL, 4.1 mol, 10 eq.) was added dropwise over a period of 1 h. After complete addition, the solution was stirred for 4 h while warming to room temperature.

Meanwhile, in a separate 500 mL recovery flask was added 3,4,5-trifluoroaniline (61 g, 0.41 mol, 1.0 eq.), conc. HCl (150 mL), and a stir bar. The resulting suspension was stirred vigorously and cooled to −10° C. The flask was fitted with a 250 mL addition funnel and a solution of NaNO₂ (34.3 g, 0.50 mol, 1.2 eq.) in H₂O (125 mL) was added to the suspension dropwise over a period of 10 min. The reaction mixture, now nearly homogeneous, is yellow-orange in color. The reaction mixture was stirred for an additional 30 min while carefully maintaining the temperature at −10° C.

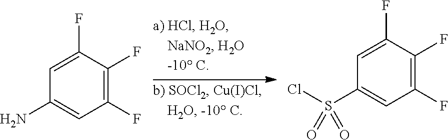

The flask containing the SOCl₂/H₂O solution is cooled again to −10° C. and a catalytic amount of Cu(I)Cl (˜50 mg) was added. The solution turns dark green in color. The flask was fitted with a 500 mL addition funnel (previously chilled to 0° C.) and the 3,4,5-trifluorodiazobenzene solution was quickly transferred to the funnel. The solution was immediately added dropwise over a period of 3 min. After addition, the reaction mixture slowly turns darker green in color, but after stirring for 5 min becomes bright, lime green. The reaction was stirred for an additional hour while warming to room temperature. The reaction mixture was transferred to a separatory funnel and extracted with CH₂Cl₂ (3×200 mL). The organic phases are combined and dried over anhydrous Na₂SO₄, filtered, and concentrated to give a dark-bronze oil (79.5 g, 83%).

### Example 5

### Enzyme Assays

mADAM-10 or hADAM-10 activity was measured as the ability to cleave a 10-residue peptide (DABCYL-Leu-Leu-Ala-Gln-Lys-*-Leu-Arg-Ser-Ser-Arg-EDANS). This peptide was based on the TNF-α cleavage site (Leu⁶²-Arg⁷¹); however, it was found that replacement of Ala⁷⁶-Val⁷⁷ with Lys-Leu resulted in a peptide with a 5-fold greater affinity for ADAM-10 than the native TNF-α peptide. Enzyme was diluted to a final active concentration of 5 nM in Buffer A (50 mM HEPES 8.0, 100 mM NaCl, 1 mM CaCl2 and 0.01% NP-40). Serial dilutions for compounds were performed ranging from 100 μM to 0.5 nM using a Beckman Biomek 2000 in polypropylene plates (Greiner). 20 μl of enzyme solution was added to 10 μl of compound in buffer A, and allowed to incubate for 15 min in 384 well black, Greiner, microtiter plates (#781076). 20 μl of substrate (12.5 μM in Buffer A) was then added, resulting in final reaction conditions of 2 nM ADAM-10, 5 μM substrate, and compound concentrations ranging from 20 uM to 0.1 nM. The reaction was incubated for 2 hr at RT, and fluorescence was measured at Ex355, Em460 on a Wallac Victor 2 fluorescence reader. For final analysis of potent inhibitors, a similar reaction was set up with a final active ADAM-10 concentration of 0.1 nM. This reaction was incubated for 16 hr at RT and fluorescence was read using identical conditions.

One aspect of the invention is, for example, piperazine-derived hydroximates according to formula I, which are selective ADAM-10 inhibitors. In one embodiment, such inhibitors comprise a bis-aryl ether substitution for —R² (—R²¹-L²-R²², where R²¹ is phenylene, L² is oxygen, and R²² is phenyl), the proximal ring (R²¹) of which is substituted particularly with one or more halogens, more particularly with one or more flourines, even more particularly with two or more flourines. For example, by combining such groups with appropriate substitution, -L¹-R¹ and -R²², inhibitors that are selective for ADAM-10 are produced.

Table 5 below shows structure activity relationship data for selected compounds of the invention when tested in vitro with various metalloproteases. Inhibition is indicated as IC₅₀ with the following key: A=IC₅₀ less than 50 nM, B=IC₅₀ greater than 50 nM, but less than 1000 nM, C=IC₅₀ greater than 1000 nM, but less than 20,000 nM, and D=IC₅₀ greater than 20,000 nM. Blank cells indicate lack of data only. The abbreviations in Table 5 are defined as follows: TACE stands for TNF-alpha converting enzyme (also known as ADAM-17; MMP-1 stands for Fibroblast collagenase; MMP-2 stands for 72 kDa gelatinase (gelatinase A); MMP-3 stands for Stromelysin-1; MMP-8 stands for Neutrophil collagenase; MMP-9 stands for 92 kDa gelatinase (gelatinase B); and MMP-13 stands for collagenase-3.

TABLE 5

| | | IC₅₀ | | | | | | | |
|---|---|---|---|---|---|---|---|---|---|
| | | ADAM- | | MMP- | MMP- | MMP- | MMP- | MMP- | MMP- |
| Entry | Structure | 10 | TACE | 1 | 2 | 3 | 8 | 9 | 13 |
| 1 | 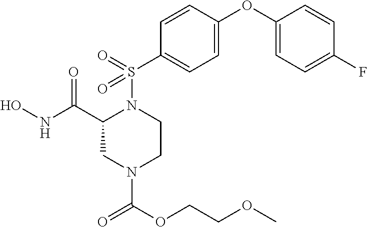 | A | | A | A | A | | | A |
| 2 | 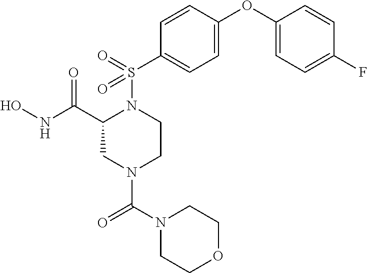 | A | | A | A | A | | | A |
| 3 | 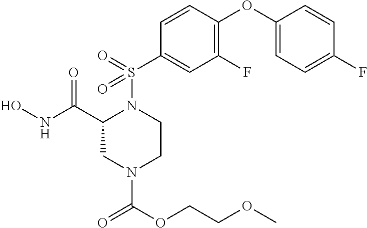 | A | | B | A | C | | | A |
| 4 | 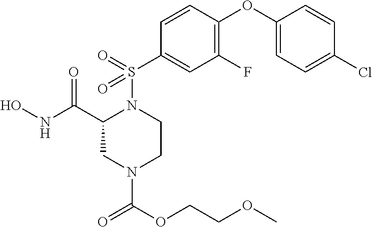 | A | | B | A | A | | | A |
| 5 | 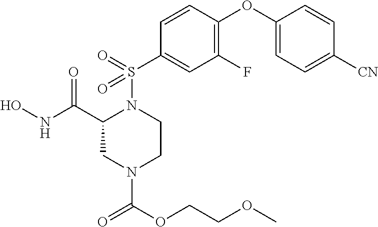 | A | | B | A | B | | | A |
| 6 | 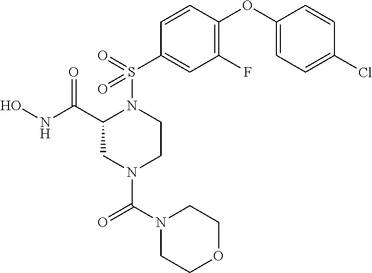 | A | | B | A | A | | | A |
| 7 | 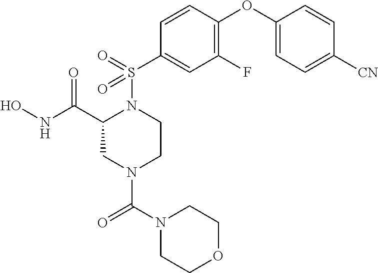 | A | | B | A | A | | | A |
| 8 | 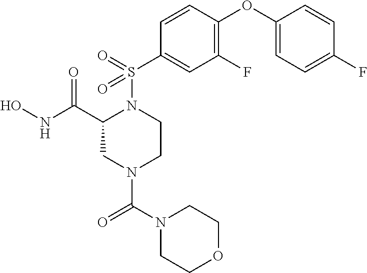 | A | | B | A | A | | | A |
| 9 | 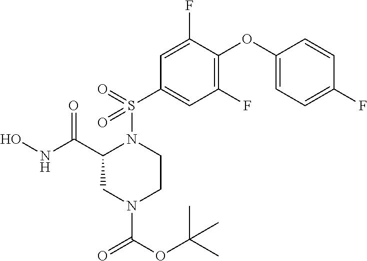 | A | | C | A | C | | | C |
| 10 | 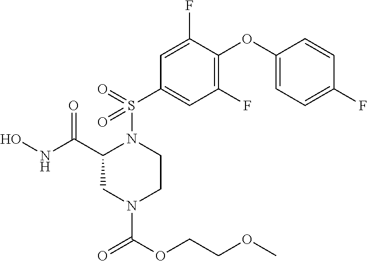 | A | | C | A | C | | | A |
| 11 | 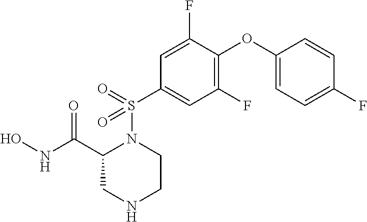 | B | | D | B | C | | | D |
| 12 | 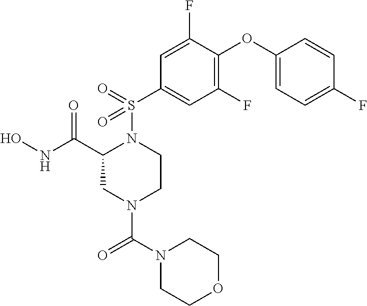 | A | | C | A | B | | | A |
| 13 | 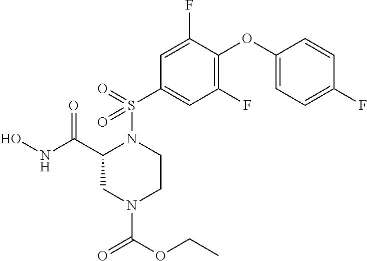 | A | | C | A | B | | | A |
| 14 | 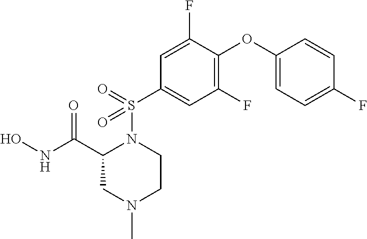 | B | | D | A | D | | | A |
| 15 | 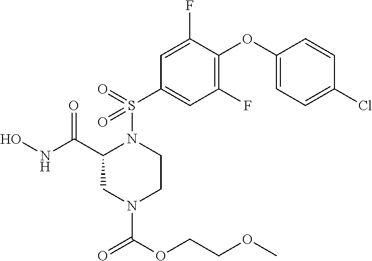 | A | B | C | A | B | A | A | A |
| 16 | 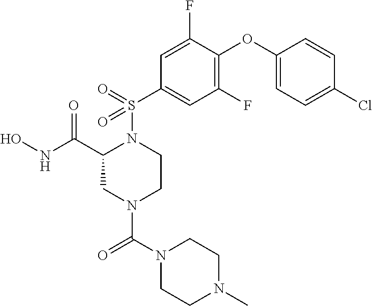 | A | | D | A | C | | | A |
| 17 | 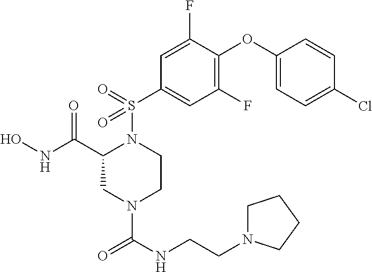 | A | | C | A | B | | | A |
| 18 | 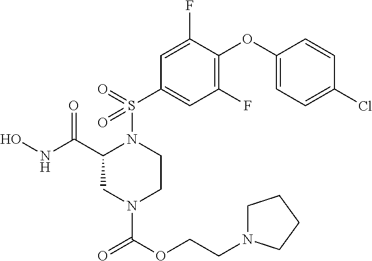 | A | | D | A | B | | | A |
| 19 | 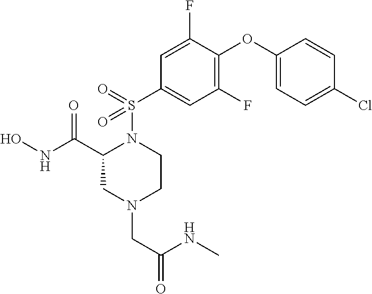 | A | | D | A | B | | | A |
| 20 | 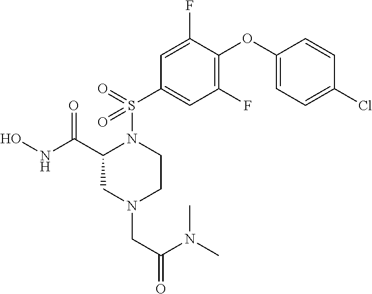 | A | | D | A | C | | | A |
| 21 | 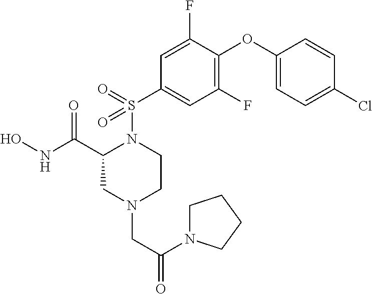 | A | | D | A | C | | | B |
| 22 | 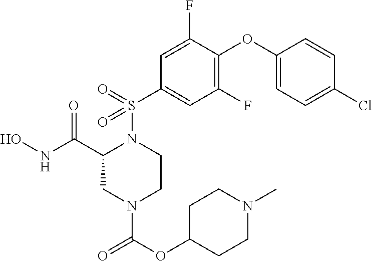 | A | | C | A | B | | | A |
| 23 | 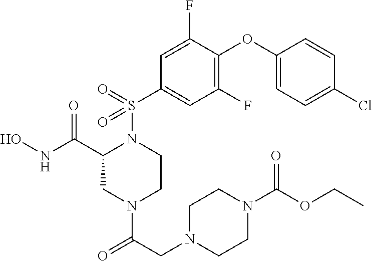 | A | | D | A | C | | | A |
| 24 | 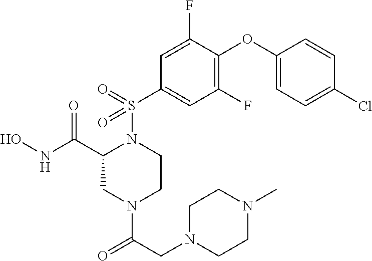 | A | | D | A | C | | | A |
| 25 | 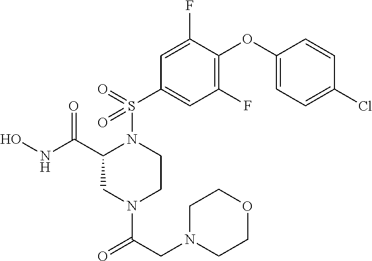 | A | | D | A | B | | | A |
| 26 | 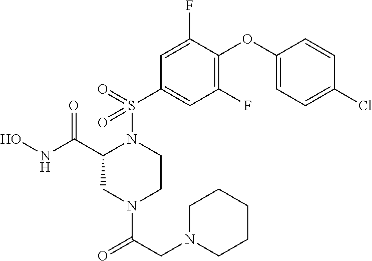 | A | | D | A | C | | | A |
| 27 | 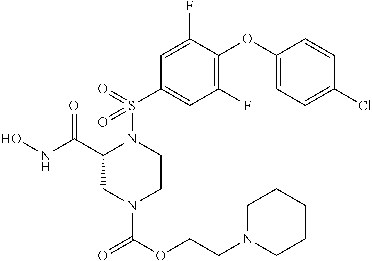 | A | | C | A | B | | | A |
| 28 | 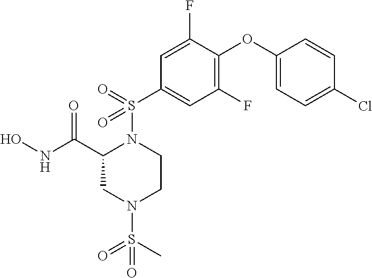 | A | | B | A | B | | | A |
| 29 | 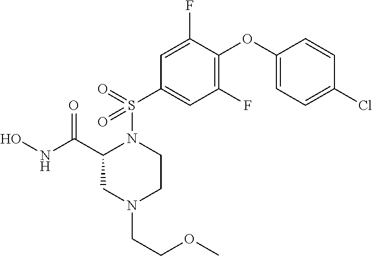 | A | | C | A | B | | | A |
| 30 | 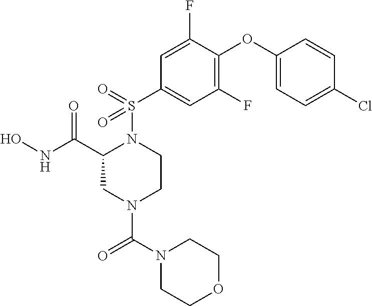 | A | B | C | A | B | A | B | A |
| 31 | 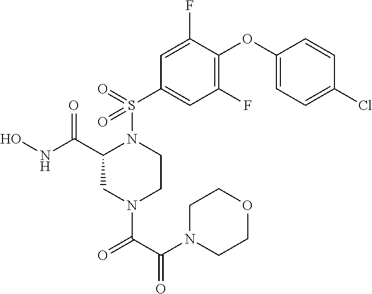 | A | B | C | A | B | | | A |
| 32 | 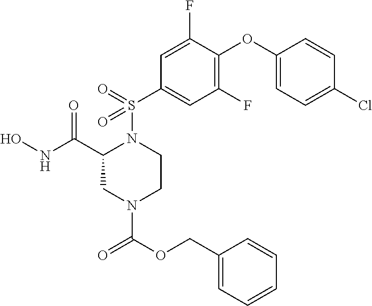 | A | | C | A | B | | | A |
| 33 | 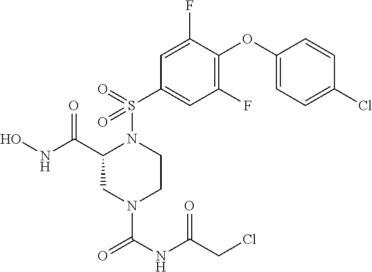 | A | | C | A | B | | | A |
| 34 | 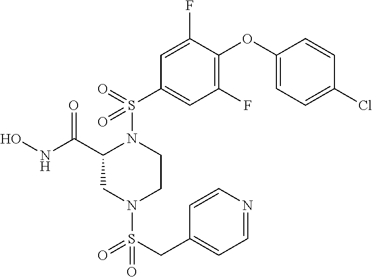 | A | A | C | A | B | | | A |
| 35 | 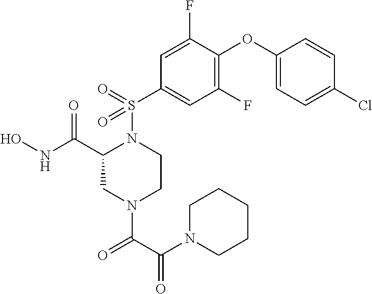 | A | | C | A | B | | | A |
| 36 | 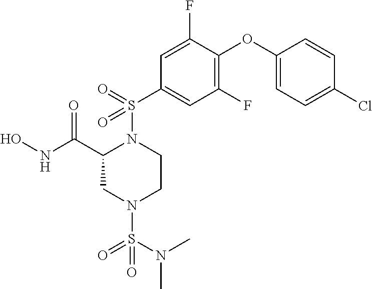 | A | | C | A | B | | | A |
| 37 | 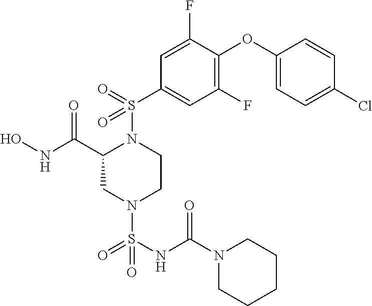 | A | B | C | A | A | | | A |
| 38 | 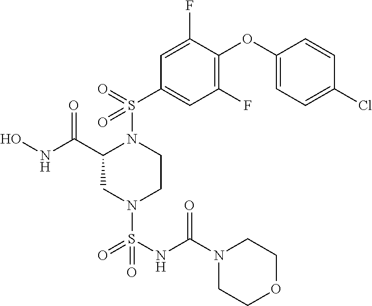 | A | B | C | A | A | | | A |
| 39 | 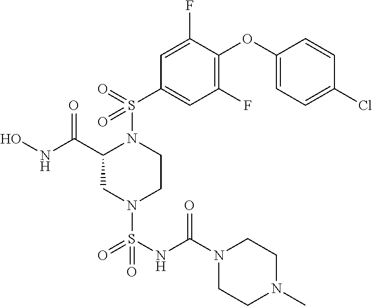 | A | | B | A | A | | | A |
| 40 | 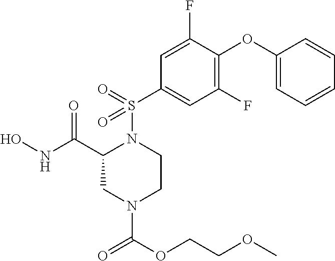 | A | | C | A | B | | | A |
| 41 | 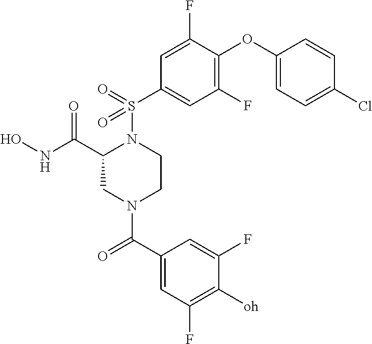 | A | | C | A | A | | | A |
| 42 | 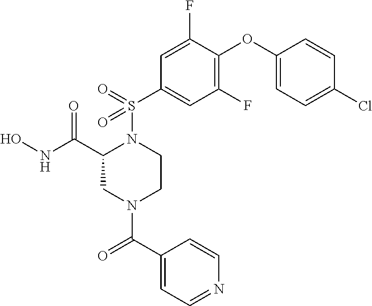 | A | | C | A | C | | | A |
| 43 | 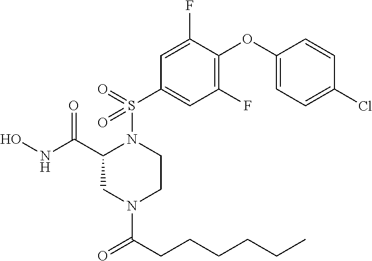 | A | | D | A | B | | | A |
| 44 | 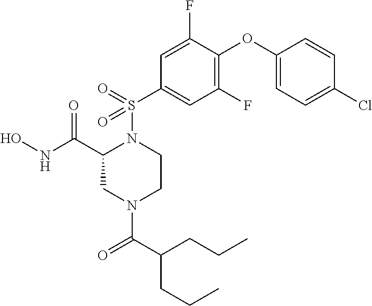 | A | | D | A | C | | | B |
| 45 | 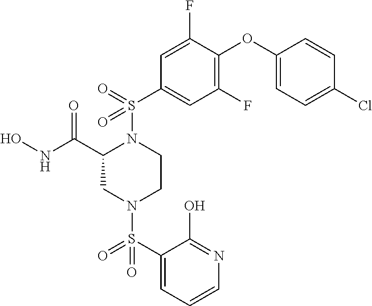 | A | B | C | A | B | | | A |
| 46 | 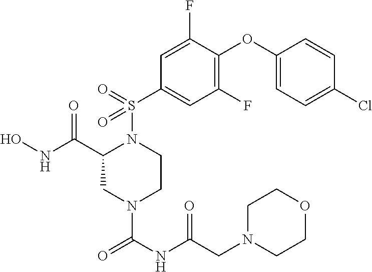 | A | | C | A | B | | | A |
| 47 | 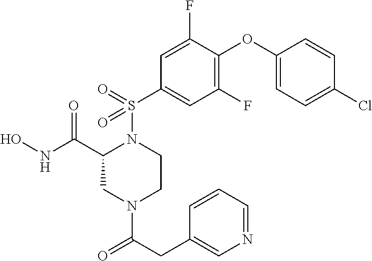 | A | | D | A | B | | | A |
| 48 | 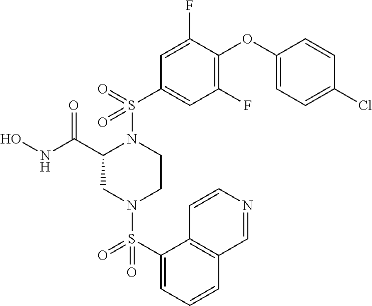 | A | | D | A | B | | | A |
| 49 | 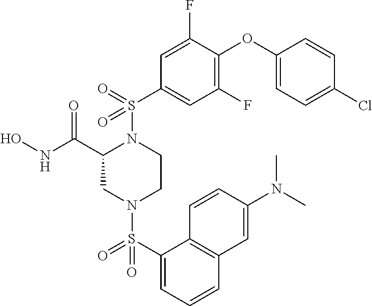 | C | | D | A | B | | | A |
| 50 | 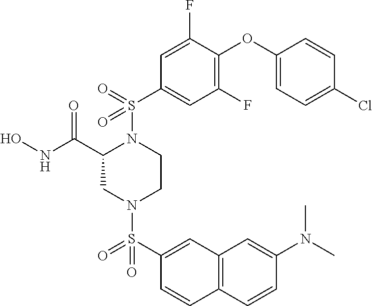 | C | | D | D | B | | | A |
| 51 | 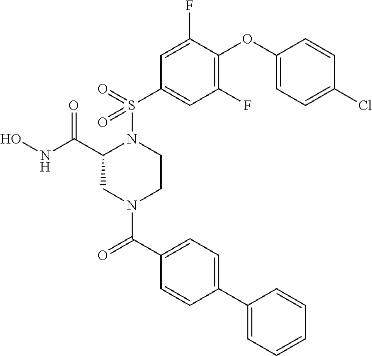 | B | | C | B | C | | | B |
| 52 | 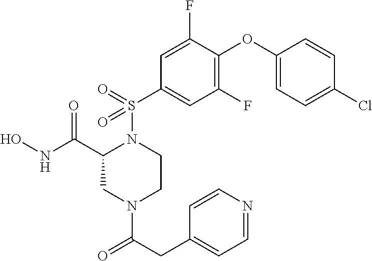 | A | | C | A | C | | | A |
| 53 | 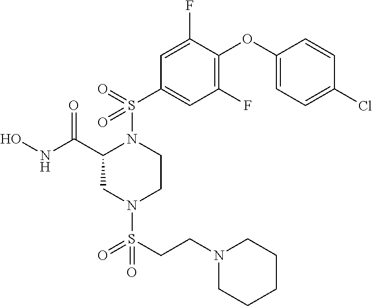 | A | | B | A | B | | | A |
| 54 | 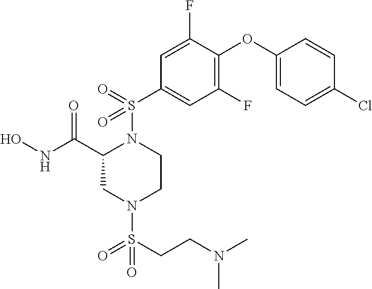 | A | A | B | A | A | | | A |
| 55 | 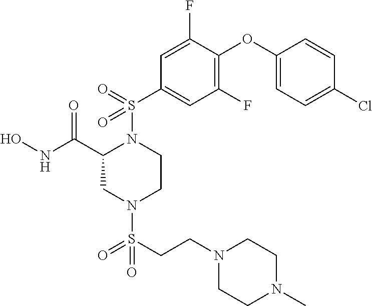 | A | | C | A | B | | | A |
| 56 | 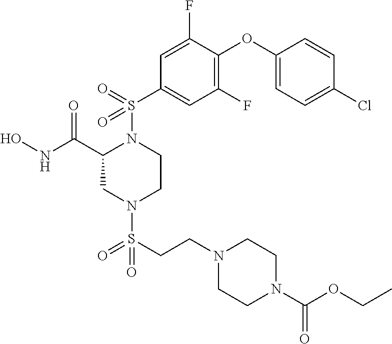 | A | | C | A | B | | | A |
| 57 | 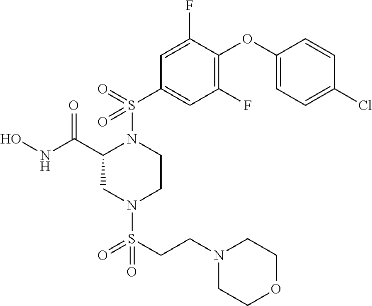 | B | | D | B | C | | | B |
| 58 | 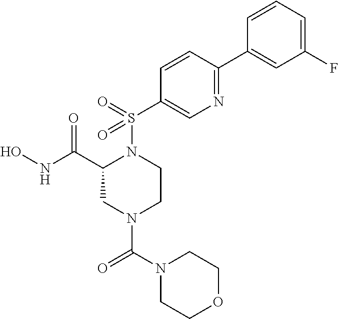 | A | | B | A | B | | | A |
| 59 | 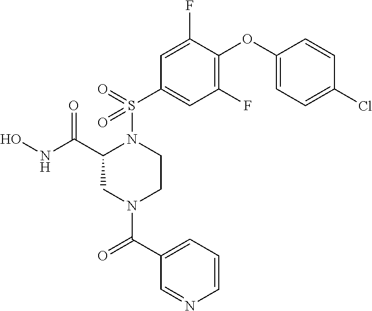 | A | B | C | A | B | | | A |
| 60 | 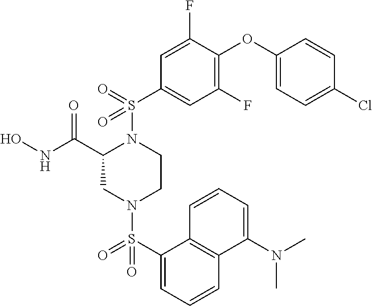 | B | | D | A | C | | | A |
| 61 | 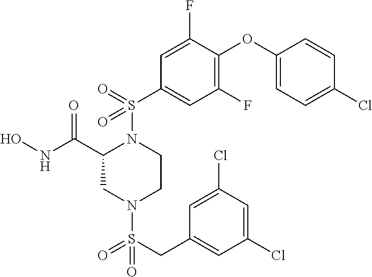 | B | | D | C | D | | | C |
| 62 | 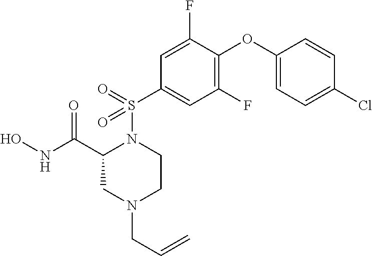 | B | | D | A | C | | | A |
| 63 | 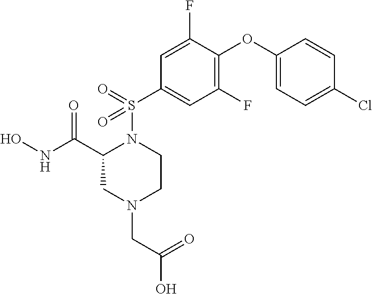 | B | | D | B | C | | | B |
| 64 | 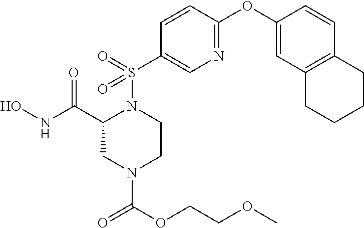 | A | | B | A | A | | | A |
| 65 | 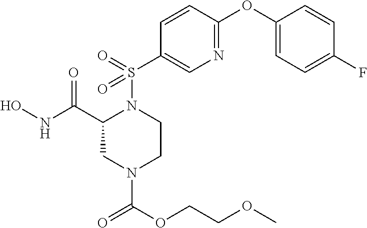 | B | | A | A | A | | | A |
| 66 | 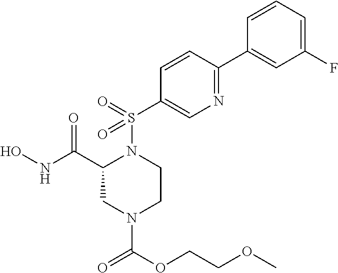 | A | | B | A | A | | | A |

Table 6 contains physical characterization data for selected compounds of the invention. ¹H-NMR data were taken with a Varian AS400 Spectrometer (400 MHz, available from Varian GmbH, Darmstadt, Germany). The entry numbers in Table 6 correspond to those of Table 5 (and their corresponding structures).

TABLE 6

| Entry | ¹H NMR Data (or MS data) |
|---|---|
| 1 | (CD3OD): 7.68 (d, 2H), 7.18-7.14 (m, 4H), 7.05 (d, 2H), 4.32 (m 1H), 4.23 (d, 1H), 4.15 |
| | (m, 2H), 4.00 (d, 1H), 3.68-3.64 (m, 2H), 3.55 (m, 2H), 3.35 (s, 3H), 3.2 (m, 1H), 3.00 (m, |
| | 1H) ppm. |
| 2 | (CD3OD): 7.69 (d, 2H, J = 9.2 Hz), 7.04 (m, 4H), 6.95 (d, 2H, J = 9.2 Hz), 4.30 (m, 1H), |
| | 3.76 (m, 1H), 3.50 (m, 7H), 3.10 (m, 4H), 2.90 (dd, 1H, J = 13.2, 4.4 Hz), 2.72 (m, 1H) |
| | ppm. |
| 3 | (CD3OD): 7.68 (dd, 1H), 7.55 (dd, 1H), 7.15-7.10 (m, 4H), 7.04 (dd, 1H), 4.28-4.12 (m, |
| | 2H), 4.15-4.00 (m, 3H), 3.70-3.65 (m, 2H), 3.55-3.50 (m, 2H), 3.33 (s, 3H), 3.22 (m, 1H), |
| | 3.03 (m, 1H) ppm. |
| 4 | (CD3OD): 7.68 (dd, 1H), 7.57 (dd, 1H), 7.38 (d, 2H), 7.13 (t, 1H), 7.08 (d, 1H), 4.28-4.12 |
| | (m, 2H), 4.15-4.00 (m, 3H), 3.70-3.65 (m, 2H), 3.55-3.50 (m, 2H), 3.33 (s, 3H), 3.22 (m, |
| | 1H), 3.03 (m, 1H) ppm. |
| 5 | (CD3OD): 7.75-7.71 (m, 3H), 7.65 (dd, 1H), 7.33 (dd, 1H), 7.20 (d, 2H), 4.32-4.26 (m, |
| | 2H), 4.16-4.05 (m, 3H), 3.81-3.75 (m, 2H), 3.56 (m, 2H), 3.34 (s, 3H), 3.27 (m, 1H), 3.06 |
| | (m, 1H) ppm. |
| 6 | (CDCl3): 7.73 (d, 1H), 7.61 (d, 1H), 7.34 (d, 2H, J = 8.8 Hz), 6.99 (d, 2H, J = 8.8 Hz), 6.98 |
| | (m, 1H), 4.67 (s, 1H), 4.23 (d, 1H), 3.64 (m, 5H), 3.44 (d, 1H), 3.35 (m, 2H), 3.21 (m, 2H), |
| | 3.10 (m, 4H) ppm. |
| 7 | (CD3OD): 7.68-7.64 (m, 3H), 7.58 (d, 1H), 7.22 (t, 1H), 7.08 (d, 2H), 4.30 (m, 1H), 3.78 |
| | (d, 1H), 3.75-3.48 (m, 7H), 3.08-3.00 (m, 5H), 2.81 (m, 1H) ppm. |
| 8 | (CD3OD): 7.75 (d, 1H), 7.60 (d, 1H), 7.18-7.14 (m, 4H), 7.07 (t, 1H), 4.4 (m, 1H), 3.86 (d, |
| | 1H), 3.78-3.55 (m, 7H), 3.24-3.14 (m, 4H), 3.08 (dd, 1H), 2.87 (m, 1H) ppm. |
| 9 | (CD3OD): 7.60-7.58 (m, 2H), 7.08-7.00 (m, 4H), 4.3-4.2 (m, 2H), 4.08-4.02 (m, 1H), |
| | 3.75-3.70 (m, 2H), 3.23-3.18 (m, 1H), 3.12-2.90 (m, 1H) ppm |
| 10 | (CD3OD): 7.49 (d, 2H), 7.08-7.00 (m, 4H), 4.3-4.2 (m, 2H), 4.18-4.05 (m, 3H), 3.75- |
| | 3.70(m, 2H), 3.55-3.50 (m, 2H), 3.33 (s, 3H), 3.33-3.25 (m, 1H), 3.15-3.00 (m, 1H) ppm. |
| 11 | (CD3OD): 7.65 (d, 2H), 7.08-6.98 (m, 4H), 4.58 (d, 1H), 4.05 (dd, 1H), 3.81 (ddd, 1H), |
| | 3.63 (d, 1H), 3.46 (d, 1H), 3.35 (dd, 1H), 3.18 (ddd, 1H) ppm. |
| 12 | (CD3OD): 7.62 (m, 2H), 7.08-7.00 (m, 4H), 4.40 (s, 1H), 3.86 (d, 1H), 3.80-3.74 (m, 2H), |
| | 3.65-3.58 (m, 5H), 3.25-3.12 (m, 5H), 2.96 (m, 1H) ppm. |
| 13 | (CD3OD): 7.60-7.58 (m, 2H), 7.08-7.00 (m, 4H), 4.3-4.2 (m, 2H), 4.08-4.02 (m, 3H), |
| | 3.75-3.70 (m, 2H), 3.27 (m, 1H), 3.05 (m, 1H) ppm. |
| 14 | (CD3OD): 7.65-7.62 (m, 2H), 7.08-7.00 (m, 4H), 4.45 (s, 1H), 3.80 (d, 1H), 3.52 (t, 1H), |
| | 3.10 (d, 1H), 2.72 (d, 1H), 2.21 (s, 3), 2.16 (d, 1H), 1.96 (t, 1H) ppm. |
| 15 | (CD3OD): 7.60 (d, 2H), 7.32 (d, 2H), 7.03 (d, 2H), 4.32-4.26 (m, 2H), 4.16-4.05 (m, 3H), |
| | 3.81-3.75 (m, 2H), 3.56 (m, 2H), 3.34 (s, 3H), 3.27 (m, 1H), 3.06 (m, 1H) ppm. |
| 16 | MS: Calculated for C23H26ClF2N5O6S: 573.13; Found: 574.72 (M + 1). |
| 17 | (CD3OD): 7.60 (d, 2H, J = 7.2 Hz), 7.32 (d, 2H, J = 8.8 Hz), 6.98 (d, 2H, J = 9.2 Hz), 4.21 |
| | (m, 2H), 4.08 (m, 1H), 3.80-3.60 (m, 5H), 3.40 (m, 1H), 3.23 (m, 2H), 3.04(m, 3H), 2.21 |
| | (m, 1H), 2.50-1.50 (m, 4H) ppm. |
| 18 | (CD3OD): 7.51 (d, 2H, J = 7.6 Hz), 7.23 (d, 2H, J = 6.4 Hz), 6.88 (d, 2H, J = 6.4 Hz), 4.19- |
| | 4.11 (m, 2H), 3.98-3.94 (m, 1H), 3.73-3.67 (m, 4H), 3.59 (m, 1H), 3.50-3.14 (m, 5H), |
| | 3.03-2.91 (m, 3H), 1.99-1.88 (m, 4H) ppm. |
| 19 | (CD3OD): 7.82 (br. s, 1H), 7.69 (d, 2H), 7.38 (d, 2H), 7.05 (d, 2H), 4.58 (br s, 1H), 3.88 |
| | (m, 1H), 3.60 (td, 1H), 3.19-2.91 (m, 4H), 2.85-2.70 (m, 6H), 2.40-2.29 (m, 2H) ppm. |
| 20 | (CD3OD): 7.71 (d, 2H), 7.35 (d, 2H), 7.00 (d, 2H), 4.58 (br s, 1H), 3.80 (m, 1H), 3.40- |
| | 3.33 (m, 2H), 3.30-3.20 (m, 2H), 3.05 (s, 3H), 2.96 (s, 3H), 2.81 (m, 1H), 2.40-2.30 (m, |
| | 2H) ppm. |
| 21 | DMSO-d₆: 9.8 (br, 1H), 9.0 (br, 1H), 7.85 (m, 2H), 7.4 (m, 2H), 7.1 (m, 2H), 4.4 (m, 3H), |
| | 3.6 (m, 7H), 3.0 (m, 3H), 2.0 (m, 4H). |
| 22 | (CD3OD): 7.61 (m, 2H), 7.32 (d, 2H, J = 8.8 Hz), 6.99 (d, 2H, J = 8.8 Hz), 4.40-4.20 (m, |
| | 4H), 4.10 (m, 1H), 3.80-3.60 (m, 4H), 3.50 (m, 1H), 3.40-3.15 (m, 4H), 2.89 (d, 3H), |
| | 2.15-2.00 (m, 2H) ppm. |
| 23 | DMSO-d₆: 10.2 (br, 1H), 9.0 (br, 1H), 7.8 (m, 2H), 7.4 (m, 2H), 7.1 (m, 2H), 4.4 (m, 4H), |
| | 4.0 (m, 7H), 3.3 (m, 8H), 1.2 (t, 3H). |
| 24 | DMSO-d₆: 7.8 (m, 2H), 7.4 (m, 2H), 7.1 (m, 2H), 3.8 (m, 11H), 3.4 (m, 2H), 3.0 (m, 4H), |
| | 2.8 (3, 3H). |
| 25 | DMSO-d₆: 10.2 (br, 1H), 9.0 (br, 1H), 7.8 (m, 2H), 7.45 (m, 2H), 7.2 (m, 2H), 4.4 (m, 4H), |
| | 3.8 (m, 7H), 3.4 (m, 6H). |
| 26 | DMSO-d₆: 9.4 (br, 1H), 9.0 (br, 1H), 7.8 (m, 2H), 7.4 (m, 2H), 7.1 (m, 2H), 4.85 (m, 1H), |
| | 4.1 (m, 2H), 3.0 (m, 6H), 3.4 (m, 4H), 3.0 (m, 2H), 1.9 (m, 4H). |
| 27 | (CD3OD): 7.54 (d, 2H, J = 7.2 Hz), 7.25 (d, 2H, J = 8.8 Hz), 6.89 (d, 2H, J = 8.8 Hz), 4.15 |
| | (m, 3H), 3.90 (m, 1H), 3.78 (m, 1H), 3.60 (m, 2H), 3.40-3.20 (m, 4H), 3.05 (m, 1H), 3.00 |
| | (m, 1H), 2.80 (m, 1H), 2.70 (m, 1H), 1.80-1.60 (m, 4H), 1.40 (m, 1H) ppm. |
| 28 | (CDCl3): 9.20 (br s, 1H), 7.58 (d, 2H), 7.30 (d, 2H), 6.90 (d, 2H), 4.65 (br s, 1H), 4.19 (d, |
| | 1H), 3.95-3.60 (m, 2H), 3.33 (m, 1H), 3.15-2.80 (m, 2H), 2.88 (s, 3H) ppm. |
| 29 | (CDCl3): 7.61 (d, 2H), 7.29 (d, 2H), 6.90 (d, 2H), 4.71 (br s, 1H), 3.75 (br d, 1H), 3.60- |
| | 3.48 (m, 2H), 3.42 (s, 3H), 3.20 (d, 1H), 3.09 (td, 1H), 2.88 (br d, 1H), 2.75 (m, 1H), |
| | 2.60-2.49 (m, 3H) ppm. |
| 30 | (CDCl3): 11.8 (br. S, 1H), 7.61 (d, 2H), 7.55 (br. s, 1H), 7.26 (d, 2H), 6.90 (d, 2H), 4.71 |
| | (s, 1H), 4.28 (d, 1H), 3.70-3.62 (m, 4H), 3.48 (d, 1H), 3.36-3.16 (m, 5H), 3.00 (t, 1H) |
| | ppm. |
| 31 | (CDCl3): 11.23 (br s, 1H), 7.59 (d, 2H), 7.26 (d, 2H), 6.95 (d, 2H), 4.70 (br s, 1H), 3.40 |
| | (br d, 1H), 4.23 (d, 1H), 3.85-3.38 (m, 10H), 3.20-2.90 (m, 2H) ppm. |
| 32 | (CDCl3): 7.46 (d, 2H, J = 6.8 Hz), 7.26 (m, 4H), 6.91 (d, 2H, J = 9.2 Hz), 4.60 (s, 1H), 4.00 |
| | (m, 1H), 3.80 (m, 2H), 3.60 (m, 2H), 3.40 (m, 1H), 2.60 (m, 2H) ppm. |
| 33 | (CDCl3): 7.54 (d, 2H, J = 5.6 Hz), 7.25 (d, 2H, J = 9.2 Hz), 6.86 (d, 2H, J = 9.2 Hz), 4.60 (m, |
| | 1H), 4.40 (m, 2H), 4.05 (m, 1H), 3.75 (m, 2H), 3.45 (m, 1H), 3.0 (m, 1H), 2.93 (s, 2H) |
| | ppm. |
| 34 | (CD3OD): 8.61 (br. s, 1H), 7.75 (m, 2H), 7.67 (d, 2H), 7.33 (d, 2H), 7.03 (d, 2H), 4.54 |
| | (m, 1H), 4.03-3.88 (m, 3H), 3.60 (m, 2H), 3.12 (m, 1H), 2.93 (m, 1H) ppm. |
| 35 | (CDCl3): 7.63 (d, 1H), 7.49 (d, 1H), 7.28 (m, 2H), 6.90 (dd, 2H), 4.51 (m, 1H), 4.42 (m, |
| | 1H), 4.14 (br d, 1H), 3.82-2.91 (m, 8H), 1.84-1.45 (m, 6H) ppm. |
| 36 | (CDCl3): 7.54 (d, 2H, J = 6.4 Hz), 7.30 (d, 2H, J = 8.8 Hz), 6.91 (d, 2H, J = 8.8 Hz), 4.70 (m, |
| | 1H), 4.10 (m, 1H), 3.90 (m, 1H), 3.60 (m, 1H), 3.40 (m, 1H), 2.83(s, 6H), 2.80 (m, 2H) |
| | ppm. |
| 37 | (CD3OD): 7.65 (d, 2H), 7.31 (d, 2H), 7.00 (d, 2H), 4.60 (m, 1H), 4.00 (m, 2H), 3.69 (m, |
| | 2H), 3.40-3.00 (m, 5H), 2.82 (m, 1H), 1.70-1.40 (m, 6H) ppm. |
| 38 | (CD3OD): 7.69 (d, 2H), 7.33 (d, 2H), 7.00 (d, 2H), 4.60 (br s, 1H), 3.92 (br t, 2H), 3.62- |
| | 3.41 (m, 10H), 2.90 (dd, 1H), 2.70 (td, 1H) ppm. |
| 39 | (CD3OD): 7.65 (d, 2H), 7.33 (d, 2H), 7.00 (d, 2H), 4.59 (br s, 1H), 3.88 (m, 2H), 3.70- |
| | 3.15 (m, 5H), 2.90-2.45 (m, 6H) ppm. |
| 40 | (CD3OD): 7.48 (d, 2H), 7.22 (dd, 2H), 6.99 (t, 1H), 6.89 (d, 2H), 4.23-4.15 (m, 2H), 4.05- |
| | 3.95 (m, 3H), 3.67-3.64 (m, 2H), 3.45 (m, 2H), 3.25 (s, 3H), 3.2 (m, 1H), 3.00 (m, 1H) |
| | ppm. |
| 41 | (CDCl3): 7.46 (d, 2H, J = 6.8 Hz), 7.26 (m, 4H), 6.91 (d, 2H, J = 9.2 Hz), 4.60 (s, 1H), 4.00 |
| | (m, 1H), 3.80 (m, 2H), 3.60 (m, 2H), 3.40 (m, 1H), 2.60 (m, 2H) ppm. |
| 42 | (CD3OD): 8.79 (br. s, 2H), 7.70 (m, 4H), 7.38 (d, 2H), 7.00 (d, 2H), 4.40 (m, 2H), 4.00- |
| | 3.00 (m, 5H) ppm. |
| 43 | (CDCl3): 7.50 (d, 2H), 7.23 (m, 2H), 6.87 (d, 2H), 4.86 (d, 1H), 4.57 (d, 1H), 4.05 (m, |
| | 2H), 3.38 (m, 2H), 3.04 (m, 1H), 2.31 (t, 2H), 1.53 (s, 2H), 1.25(s, 6H), 0.85(t, 3H) ppm. |
| 44 | (CDCl3): 7.52 (d, 2H, J = 6.4 Hz), 7.24 (d, 2H, J = 8.8 Hz), 6.87 (d, 2H, J = 8.4 Hz), 4.97 (d, |
| | 1H), 4.71 (s, 1H), 4.05 (d, 1H), 3.80 (d, 1H), 3.37 (m, 1H), 3.26 (t, 1H), 3.05(d, 1H), 2.62 |
| | (m, 1H), 1.54(m, 2H), 1.80(m, 2H), 1.18(m, 4H), 0.85(dt, 6H) ppm. |
| 45 | (CDCl3): 8.15 (s, 1H), 7.65 (s, 1H), 7.47 (m, 2H), 7.21 (d, 2H, J = 8.8 Hz), 6.84 (d, 2H, |
| | J = 8.4 Hz), 6.43 (s, 1H), 4.63 (s, 1H), 3.60 (m, 3H), 2.80 (m, 3H) ppm. |
| 46 | MS: Calculated for C24H26ClF2N5O8S: 617.12; Found: LC/MS: 618.2 (M + 1). |
| 47 | (CD3OD): 8.60 (m, 2H), 8.25 (d, 1H), 7.83 (m, 1H), 7.62-7.50 (m, 2H), 7.22 (m, 2H), |
| | 6.85 (m, 2H), 4.60-4.20 (m, 2H), 4.15-3.95 (m, 2H), 3.85-3.65 (m, 2H), 3.50-3.40 (m, |
| | 2H), 3.10 (m, 1H) ppm. |
| 48 | (CD3OD): 9.60 (br s, 1H), 8.60 (m, 4H), 7.95 (t, 1H), 7.60 (d, 2H), 7.37 (d, 2H), 7.00 (d, |
| | 2H), 4.60 (br s, 1H), 4.15 (br d, 1H), 3.93 (br d, 1H), 3.71-3.42 (m, 2H), 2.80-2.50 (m, |
| | 2H) ppm. |
| 49 | (CD3OD): 8.50 (d, 1H), 7.99 (d, 1H), 7.79 (d, 1H), 7.58 (m, 2H), 7.40 (m, 4H), 7.11 (m, |
| | 3H), 4.60 (br s, 1H), 4.20 (br d, 1H), 3.85 (br d, 1H), 3.49 (m, 2H), 3.09 (s, 6H), 2.50 (dd, |
| | 1H), 2.30 (td, 1H) ppm. |
| 50 | (CD3OD): 8.09 (s, 1H), 7.80 (dd, 2H), 7.60-7.42 (m, 3H), 7.31 (m, 3H), 7.95 (m, 3H), |
| | 4.60 (br s, 1H), 4.08 (m, 1H), 3.91 (br d, 1H), 3.60 (m, 2H), 3.10 (s, 6H), 2.42 (dd, 1H), |
| | 2.22 (td, 1H) ppm. |
| 51 | (CDCl3): 7.63 (d, 2H, J = 7.6 Hz), 7.56(d, 2H, 7.2 Hz), 7.53-7.37 (m, 6H), 7.24 (m, 3H), |
| | 6.86 (d, 2H, J = 8.8 Hz), 3.90 (s, 1H), 3.70 (m, 2H), 3.45 (m, 1H), 3.30 (m, 3H) ppm. |
| 52 | (CD3OD): 8.45 (br s, 2H), 7.78 (d, 1H), 7.58 (m, 3H), 7.38 (m, 2H), 7.00 (m, 2H), 4.80- |
| | 4.05 (m, 2H), 4.00-3.77 (m, 5H), 3.45-3.05 (m, 2H) ppm. |
| 53 | (CD3OD): 7.70 (d, 2H), 7.39 (d, 2H), 7.00 (d, 2H), 4.60 (br s, 1H), 4.00 (m, 2H), 3.79 (m, |
| | 2H), 4.60-3.40 (m, 6H), 3.20-2.90 (m, 4H), 2.00-1.40 (m, 6H) ppm. |
| 54 | (CD3OD): 7.70 (d, 2H), 7.39 (d, 2H), 7.00 (d, 2H), 4.60 (br s, 1H), 4.00 (m, 2H), 3.75 (m, |
| | 2H), 4.49 (m, 4H), 3.18 (m, 2H), 2.93 (s, 6H) ppm. |
| 55 | (CD3OD): 7.66 (d, 2H), 7.35 (d, 2H), 7.03 (d, 2H), 4.58 (m, 1H), 4.03-3.92 (m, 3H), 3.71- |
| | 3.68 (m, 3H), 3.27-3.25 (t, 2H), 3.15-3.13 (m, 4H), 2.97-2.93 (m, 1H), 2.88 (s, 3H), 2.86- |
| | 2.82 (m, 5H) ppm |
| 56 | (CD3OD): 7.68-7.66 (d, 2H), 7.35-7.33 (d, 2H), 7.04-7.01(d, 2H), 4.57 (m, 1H), 4.13-4.08 |
| | (q, 2H), 4.02-3.98 (m, 1H), 3.71-3.68 (m, 2H), 3.46 (m, 4H), 3.26-3.23 (t, 2H), 3.19-3.15 |
| | (dd, 1H), 2.96-2.95 (m, 1H), 2.77-2.73 (m, 2H), 2.46 (m, 4H), 1.26-1.22 (t, 3H) ppm |
| 57 | (CD3OD): 7.19 (d, 2H), 7.14 (d, 2H), 6.83 (d, 2H), 4.48 (br s, 1H), 3.95-3.92 (br d, 1H), |
| | 3.83-3.80 (br d, 1H), 3.58-3.53 (m, 6H), 3.15 (dd, 2H), 2.94 (dd, 1H), 2.75-2.74 (td, 1H), |
| | 2.63-2.60 (t, 2H), 2.40-2.39 (m, 4H) ppm |
| 58 | (CD3OD): 9.00 (d, 1H), 8.23 (d, 1H), 8.07 (d, 1H), 7.92-7.86 (m, 2H), 7.52 (m, 1H), 7.22 |
| | (m, 1H), 4.50 (m, 1H), 3.90-3.57 (m, 8H), 3.22-3.08 (m, 5H), 2.97 (m, 1H) ppm. |
| 59 | (CD3OD): 8.54 (d, 2H), 7.77 (br s, 1H), 7.57-7.50 (m, 2H), 7.44-7.42 (m, 1H), 7.27-7.22 |
| | (m, 2H), 6.95-6.92 (m, 2H), 4.40-4.20 (m, 1H), 3.85-3.60 (m, 3H), 3.57-3.18 (m, 2H), |
| | 3.10-2.95 (m, 1H) ppm |
| 60 | MS: calculate for C29H27ClF2N4O7S2: 680.10; found: 681.20 (M + 1). |
| 61 | MS: calculated for C24H20Cl3F2N3O7S2: 668.98; found: 669.90 (M + 1). |
| 62 | (CD3OD): 7.63 (d, 2H, J = 7.2 Hz), 7.25 (d, 2H, J = 9.2 Hz), 6.93 (d, 2H, J = 9.2 Hz), 5.79 |
| | (m, 1H), 5.47 (s, 1H), 5.44(d, 1H), 4.56 (d, 1H), 4.00 (d, 1H), 3.70-3.50 (m, 4H), 3.35 (d, |
| | 1H), 2.99 (d, 1H), 2.88 (t, 1H) ppm. |
| 63 | (CD3OD): 7.66 (d, 2H, J = 7.6 Hz), 7.35 (d, 2H, J = 8.8 Hz), 6.99 (d, 2H, J = 9.2 Hz), 3.85 (d, |
| | 1H), 3.67 (s, 2H), 3.61 (d, 1H), 3.44 (m, 2H), 3.04 (d, 1H), 2.83 (dd, 1H), 2.66 (dt, 1H) |
| | ppm. |
| 64 | (CD3OD): 8.45 (d, 1H), 8.10 (dd, 1H), 7.12 (d, 1H), 7.02 (d, 1H), 6.86-6.82 (m, 2H), 4.33- |
| | 4.25 (m, 2H), 4.15-4.05 (m, 3H), 3.70-3.65 (m, 2H), 3.55 (m, 2H), 3.35 (s, 3H), 3.25 (m, |
| | 1H), 3.05 (m, 1H), 2.78 (m, 4H), 1.80 (m, 4H) ppm. |
| 65 | (CD3OD): 8.47 (d, 1H), 8.12 (dd, 1H), 7.22-7.09 (m, 5H), 4.33-4.25 (m, 2H), 4.15-4.05 (m, |
| | 3H), 3.70-3.65 (m, 2H), 3.55 (m, 2H), 3.33 (s, 3H), 3.25 (m, 1H), 3.05 (m, 1H) ppm. |
| 66 | (CD3OD): 9.96 (d, 1H), 8.20 (d, 1H), 8.14 (d, 1H), 7.90 (d, 1H), 7.86 (d, 1H), 7.50 (m, |
| | 1H), 7.21 (m, 1H), 4.40 (m, 1H), 4.28 (d, 1H), 4.12-4.05 (m, 3H), 3.75-3.70 (m, 2H), 3.52 |
| | (m, 2H), 3.30 (s, 3H), 3.25 (m, 1H), 3.06 (m, 1H) ppm. |

### Example 6

### In Vivo Assays

MMP inhibitors of the invention were evaluated in a well-established mouse model (standard elastase-perfusion model) of AAA to determine effectiveness relative to treatment with doxycycline (which has been shown to effectively inhibit model aneurysm development via inhibition of MMP activity). All mice used in the experiment were commercially obtained C57/B16 inbred strain mice. Throughout the experimental course, mice were allowed free access to food and water. Animals were housed in a controlled animal facility, and all mouse care and treatment occurred under approved protocols.

### Elastase Perfusion Model:

A total of 89 C57/B16 mice were subjected to transient perfusion of the abdominal aorta according to a protocol known in the art. Briefly, after sedation and preparation, the aorta was approached through a midline laparotomy. The infrarenal aorta was dissected and the diameter was measured under physiologic blood pressure. A segment of infrarenal aorta was isolated and a 5 minute perfusion of this segment was performed through an arteriotomy at 100 mmHg with a solution containing type I porcine pancreatic elastase (PPE 0.16 U/mL). All of the experiments were performed with a single PPE preparation derived from the same commercial source and lot.

Following aortic perfusion the arteriotomy was repaired, the laparotomy was closed and the animal was allowed to completely recover before returning to its standard housing.

### Experimental Treatment:

Following aortic perfusion, animals were placed into one of 5 treatment groups. All animals treated with the experimental agent (Compound 15 as shown in Table 5), received gavage daily with the agent diluted in Cremophor, a non-ionic castor oil-based solubilizer and emulsifying agent (BASF). Three different doses of the agent were used, 50 mg/kg/day (n=17), 125 mg/kg/day (n=17), and 250 mg/kg/day (n=18). There were two mice in each group which died following aortic perfusion, and all others were available for analysis. Control animals (n=18) were similarly treated with daily gavage of the Cremophor diluent only. Of these mice, 16 survived the two weeks following aortic perfusion and underwent final aortic measurements and harvest. The fifth group of mice did not receive a gavage treatment, but were treated with doxycycline in their drinking water at a concentration intended to deliver 100 mg/kg/day based on the known water consumption of the animals. In this group, 4 mice died prior to the two week harvest, and all others were used in the analysis of aneurysm growth.

### Final Aortic Diameter Measurement and Specimen Collection:

Two weeks following elastase perfusion, the mice were again anesthetized; the laparotomy incision was reopened and the final aortic diameter was measured in vivo prior to sacrifice. The animals were humanely sacrificed, and circulating blood and the entire perfused segment of aorta was harvested for RNA extraction, protein extraction or histology.

### Light Microscopy:

The aortas from several mice from each experimental group were perfusion fixed with 10% neutral-buffered formalin, removed, and placed in additional formalin for a minimum of 24 hours prior to processing for paraffin embedding. Following paraffin embedding, aortic specimens were cut into 5 μm sections and mounted on glass slides. Each specimen was stained with hematoxylin and eosin to evaluate inflammatory cell infiltration and Accustain® Elastic Stain kit to assess the degree of elastin degradation. Photomicrographs of serial sections were obtained using an Olympus BX60 light microscope equipped with CV12 video capture camera.

### Results

### Effects of Compound 15 on Aortic Diameter at Harvest:

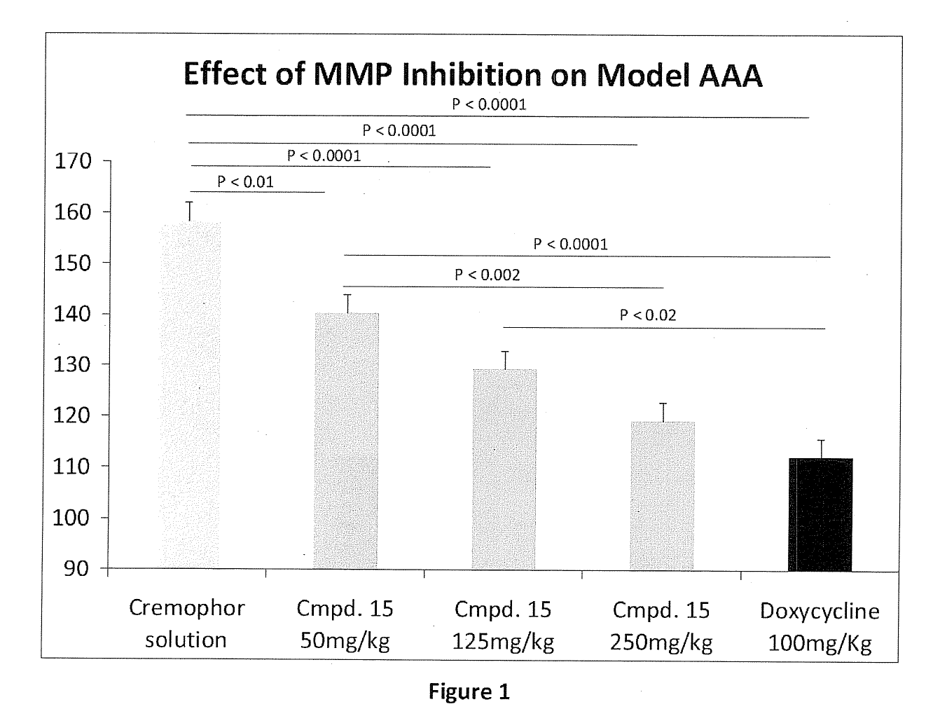

Results are expressed as the percentage increase in aortic diameter (AD) at 2 weeks compared to baseline (% ΔAD). In control animals (n=16) which only received twice daily gavage with the carrier, cremophor solution, the mean % ΔAD was 158.5±4.3% (FIG. 1), and all of the animals had a % ΔAD which was greater than 100% (the definition of aneurysm development in this model). Treatment with doxycycline (n=15) resulted in a mean % ΔAD significantly less than the control animals at 112.2±2.0% (P<0.0001). Doxycycline treatment also resulted in 13% of animals not reaching the threshold designated for aneurysms in the model. This difference did not reach statistical significance compared to the control.

All doses of treatment with the experimental agent were found to result in aortic diameters at harvest which were significantly smaller than control animals. The treatment was found to have a dose response relationship. Animals treated with the highest dose of the experimental agent (250 mg/kg/day) were found to have an increase in aortic diameter significantly less than control animals (119.2±14.1%, P<0.0001) and not significantly different than the doxycycline treated animals. There were 12% of animals which did not develop aneurysms—similar to that seen with doxycycline treatment, but not statistically different than controls.

Treatment of the animals with the lower doses of agent resulted in larger diameters of aortas at harvest. Treatment with the experimental agent at 1251 ng/kg/day resulted in a mean % ΔAD of 129.3±5.1% which was significantly less than control mice (P<0.0001), but also was greater than doxycycline treatment (P<0.02). Similarly, treatment with the lowest dose of the agent resulted in a mean % ΔAD of 140.4±3.2%, which while being significantly smaller than control treatment (P<0.01) was greater than treatment with either doxycycline (P<0.0001) or the highest dose of the experimental agent (P<0.002). All animals in both the low and intermediate experimental agent dose groups developed maximal diameters greater than 100%.

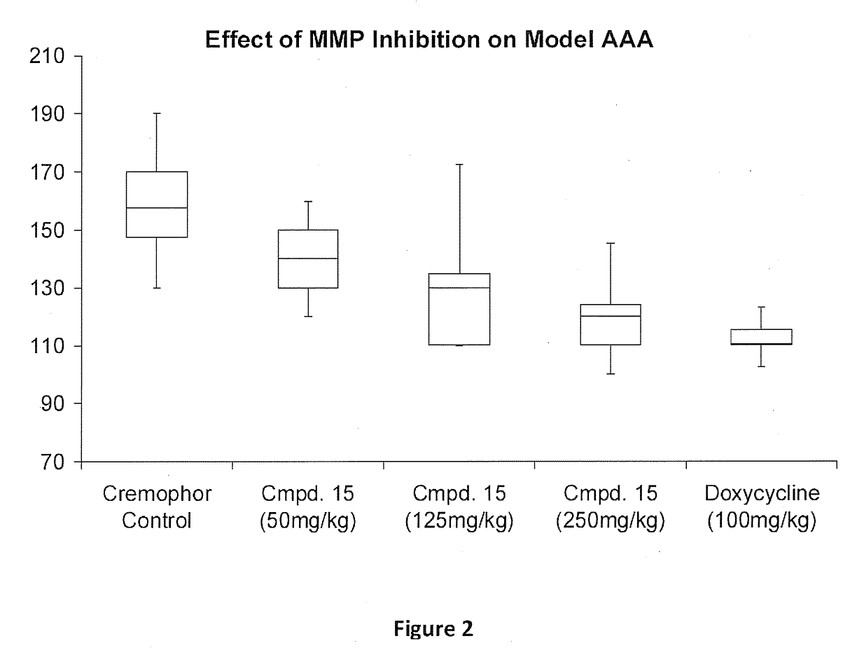

FIG. 2 shows a box-and-whisker plot. The median % ΔAD diminished with increasing experimental agent dosage. The variability of the results for each treatment was rather small. Only one animal treated with the experimental agent (at the 125 mg/kg dose) had a % ΔAD of greater than the median of the control animals. The results also do not show evidence of reaching the maximal effect of the agent at the highest dosage used in this study.

### Aortic Histology:

Representative aortas from each group were fixed in formalin following aortic harvest and processed into paraffin blocks. During harvest only the maximally dilated segment of the aorta was taken, and serial sections of the block were made to assure that the most dilated segment of aorta was imaged. These maximally dilated segments were stained with Hematoxylin and Eosin stains as well as an elastin highlighting stain (Verhoff-Von Giesen [VVG]).

In the absence of MMP-inhibitor therapy, aortas from control animals showed severe medial elastic fiber destruction associated with an appreciable mononuclear cellular infiltration. Treatment with doxycycline following elastase perfusion resulted in preservation of the medial elastin, but there continued to be a modest cellular infiltrate. With treatment with the experimental agent, the degree of elastin damage and inflammatory cell inflammation inversely correlated with the dose of the agent administered. As the mean dilatation of the aorta increased there was more extensive destruction of the elastic fibers which also appears associated with a more extensive inflammatory cell infiltrate, particularly within the adventitia.

1. A method of treating aneurysmal dilatation or blood vessel wall weakness, comprising administering to a mammal in need of such treatment a therapeutically effective amount of (a) a compound, wherein the compound is 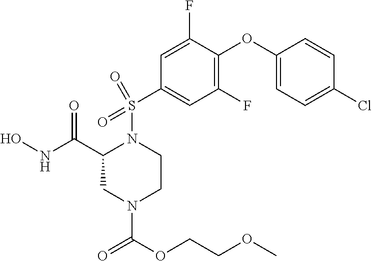
or a pharmaceutically acceptable salt thereof, or (b) a composition comprising the compound and a pharmaceutically acceptable carrier.

2. (canceled)

3. A method of modulating the activity of an MMP comprising administering to a mammal in need of such modulation an effective MMP-modulating amount of (a) a compound, wherein the compound is 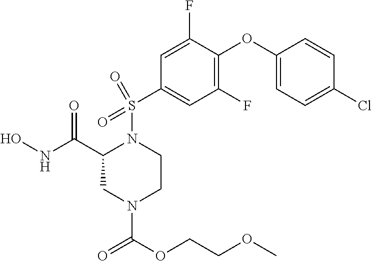
or a pharmaceutically acceptable salt thereof, or (b) a composition comprising the compound and a pharmaceutically acceptable carrier.

4. A method of treating aneurysmal dilatation or blood vessel wall weakness, comprising administering to a mammal in need of such treatment a therapeutically effective amount of an MMP inhibitor in conjunction with an angiotensin converting enzyme inhibitor, an angiotensin II receptor blocker, or a cyclophilin A inhibitor.

5. The method of claim 4, wherein the angiotensin converting enzyme inhibitor is selected from one of the following: captopril, zofenopril, enalapril, ramipril, quinapril, perindopril, lisinopril, benazepril, and fosinopril.

6. (canceled)

7. The method of claim 4, wherein the angiotensin II receptor blocker is selected from one of the following: candesartan, aprosartan, irbesartan, valsartan, and losartan.

8. (canceled)

9. The method of claim 4, wherein the MMP inhibitor is a compound, wherein the compound is 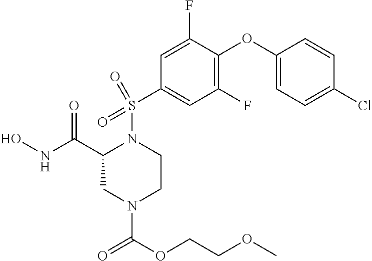
or a pharmaceutically acceptable salt thereof, which is administered in conjunction with an angiotensin converting enzyme inhibitor.

10. The method of claim 4 wherein the MMP inhibitor is a compound, wherein the compound is 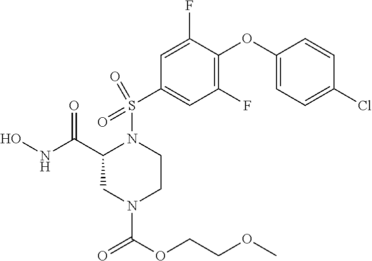
or a pharmaceutically acceptable salt thereof, which is administered in conjunction with an angiotensin II receptor blocker.

11. A method of treating aneurysmal dilatation or blood vessel wall weakness, comprising administering to a mammal in need of such treatment a therapeutically effective amount of (a) a compound, wherein the compound is 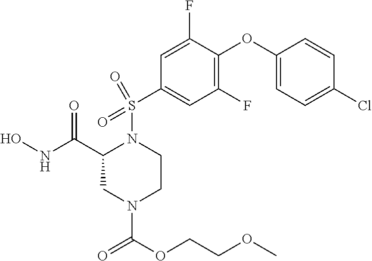
or a pharmaceutically acceptable salt thereof, or (b) a pharmaceutical composition comprising the compound and a pharmaceutically acceptable carrier;
in conjunction with an angiotensin II receptor blocker and an inhibitor of angiotensin converting enzyme.

12. The method of claim 4 wherein the MMP inhibitor is a compound of the structure: 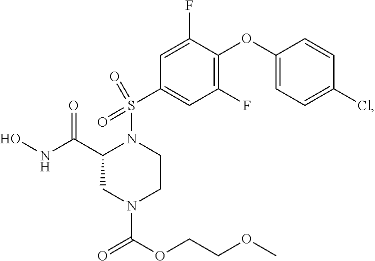
which is administered in conjunction with a cyclophilin A inhibitor.

13. The method of claim 1, wherein the aneurysmal dilatation or blood vessel wall weakness is an abdominal aortic aneurysm or a thoracic aneurysm.

14. The method of claim 3, wherein the aneurysmal dilatation or blood vessel wall weakness is an abdominal aortic aneurysm or a thoracic aneurysm.

15. The method of claim 4, wherein the aneurysmal dilatation or blood vessel wall weakness is an abdominal aortic aneurysm or a thoracic aneurysm.

16. The method of claim 9, wherein the aneurysmal dilatation or blood vessel wall weakness is an abdominal aortic aneurysm or a thoracic aneurysm.

17. The method of claim 10, wherein the aneurysmal dilatation or blood vessel wall weakness is an abdominal aortic aneurysm or a thoracic aneurysm.

18. The method of claim 11, wherein the aneurysmal dilatation or blood vessel wall weakness is an abdominal aortic aneurysm or a thoracic aneurysm.

19. The method of claim 12, wherein the aneurysmal dilatation or blood vessel wall weakness is an abdominal aortic aneurysm or a thoracic aneurysm.
